(19) United States
(12) Patent Application Publication
(10) Pub. No.: US 2026/0345171 A1
(43) Pub. Date: Nov. 5, 2026

(54) VASO-OCCLUSIVE DEVICES FOR OCCLUDING BLOOD VESSELS AND METHODS FOR MAKING AND USING SAME

(71) Applicants: STRYKER CORPORATION, Portage, MI (US); STRYKER EUROPEAN OPERATIONS LIMITED, Carrigtwohill (IE)

(72) Inventors: Esther Koo, Portage, MI (US); Clifford Teoh, Los Altos, CA (US); Stella Than Chu, Portage, MI (US); Andrew S. Lee, San Jose, CA (US); Hancun Chen, San Ramon, CA (US); Randy Gamarra, Portage, MI (US); Kevin Tran, Portage, MI (US); Alyssa Meyer, Portage, MI (US); Jason Delgadillo, Portage, MI (US)

(73) Assignees: STRYKER CORPORATION, Portage, MI (US); STRYKER EUROPEAN OPERATIONS LIMITED, Carrigtwohill (IE)

(21) Appl. No.: 19/195,663

(22) Filed: Apr. 30, 2025

(51) Int. Cl. A61B 17/12

(52) U.S. Cl. CPC A61B 17/12113; A61B 17/12145; A61B 2017/1205

(57) ABSTRACT

A vaso-occlusive device includes: an elongated member configured to form a three-dimensional structure when deployed outside a delivery catheter; wherein the three-dimensional structure comprises a first portion having first loops, and a second portion having second loops; wherein the first loops of the first portion comprise a first set of at least three first loops lying within at least three different respective first planes that are non-parallel and non-perpendicular with respect to each other; and wherein the second loops of the second portion comprise a second set of at least three second loops lying within at least three different respective second planes that are non-parallel and non-perpendicular with respect to each other.

### FIELD

The present disclosure relates generally to medical devices and intravascular medical procedures and, more particularly, to devices for occluding vasculature, such as blood vessels, and methods for making and using such devices.

### BACKGROUND

Vaso-occlusive devices or implants are used for a wide variety of reasons, including treatment of intra-vascular aneurysms. An aneurysm is a dilation of a vessel, such as a blood vessel, that may pose a risk to a patient's health due to rupture, clotting, or dissection. For example, rupture of an aneurysm in a patient's brain may cause a stroke, and lead to brain damage and death. Cerebral aneurysms may be detected in a patient, e.g., following seizure or hemorrhage, and may be treated by applying vaso-occlusive devices.

Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., which is fully incorporated herein by reference as though set forth in full, describes a vaso-occlusive device that assumes a linear, helical primary shape when stretched for placement through the lumen of a delivery catheter, and a folded, convoluted secondary shape when released from the delivery catheter and deposited in the vasculature. In order to better frame and fill aneurysms, complex three-dimensional secondary shapes can be imparted on vaso-occlusive devices and the stiffness/flexibility of vaso-occlusive devices can be modified.

In order to deliver the vaso-occlusive devices to a desired site in the vasculature, e.g., within an aneurysmal sac, it is known to first position a small profile delivery catheter or “micro-catheter” at the site using a guidewire. Typically, the distal end of the micro-catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysmal sac once the guidewire is withdrawn. A delivery or “pusher” assembly or “wire” is then passed through the micro-catheter until a vaso-occlusive device coupled to a distal end of the delivery assembly is extended out of the distal end opening of the micro-catheter and into the aneurysmal sac. Once in the aneurysmal sac, portions of the vaso-occlusive device may deform or bend to allow more efficient and complete packing. The vaso-occlusive device is then released or “detached” from the distal end of the delivery assembly, and the delivery assembly is withdrawn back through the micro-catheter. Depending on the particular needs of the patient, one or more additional vaso-occlusive devices may be pushed through the micro-catheter and released into the same aneurysmal sac.

Fluoroscopy is typically used to visualize vaso-occlusive devices during delivery into an aneurysm, while magnetic resonance imaging (MRI) is typically used to visualize the treatment site post-procedure (e.g., a few weeks after initial treatment of the aneurysm) to ensure that the aneurysmal sac is properly occluded. As such, it is important that vaso-occlusive devices be constructed in a manner that enables their radiopacity during treatment of the aneurysm, while minimizing any visualization obscuring artifacts created during the post-procedure MRI (i.e., being MRI-compatible). It is also preferable that such vaso-occlusive devices be “soft” (i.e., be laterally flexible or conformable), and thus atraumatic, to prevent rupturing of the delicate tissues of the aneurysm.

In some cases, vaso-occlusive devices are utilized to occlude blood vessels. In such cases, physicians may use vaso-occlusive devices designed for treating aneurysms to occlude blood vessels. However, because blood vessels and aneurysms have different anatomical shapes, use of aneurysm vaso-occlusive devices for blood vessel treatment may not provide adequate treatment outcome. For example, because aneurysm vaso-occlusive devices are designed to occupy a small, isolated space inside the aneurysm, they may not have the sufficient implant length to occlude a blood vessel. As a result, the physician may use multiple aneurysm vaso-occlusive devices to occlude a blood vessel, thereby lengthening a procedure time. Because the aneurysm vaso-occlusive devices do not have a shape to sufficiently anchor the vaso-occlusive devices against the wall of the blood vessel, the delivered vaso-occlusive devices may become loose and may migrate along the blood vessel after implantation.

Thus, new vaso-occlusive devices that address the above concerns are desirable.

### SUMMARY

A vaso-occlusive device includes: an elongated member having a primary configuration when constrained inside a delivery catheter; wherein the elongated member is configured to form a three-dimensional structure having a secondary configuration when deployed outside the delivery catheter; wherein the three-dimensional structure comprises a first portion having first loops, and a second portion having second loops; wherein the first loops of the first portion comprise a first set of at least three first loops lying within at least three different respective first planes that are non-parallel and non-perpendicular with respect to each other; and wherein the second loops of the second portion comprise a second set of at least three second loops lying within at least three different respective second planes that are non-parallel and non-perpendicular with respect to each other.

Optionally, the three-dimensional structure also comprises a first helical section disposed between the first portion and the second portion, wherein the first helical section comprises at least 1.0 turns.

Optionally, the first planes form a pyramidal shape.

Optionally, the pyramidal shape comprises a polyhedral shape formed of a polygonal base having n number of sides and n number of lateral faces connecting to an apex.

Optionally, the three-dimensional structure comprises a second helical section extending from the second portion.

Optionally, the second helical section has a longitudinal length that is longer than a cross-sectional width of the second portion of the three-dimensional structure.

Optionally, the elongated member is a primary coil.

Optionally, the first helical section comprises a first secondary coil formed by a first segment of the primary coil, wherein the first portion is formed by a second segment of the primary coil, and the second portion is formed by a third segment of the primary coil.

Optionally, the three-dimensional structure comprises a first anchor at a distal end of the vaso-occlusive device.

Optionally, the first anchor comprises a second helical section.

Optionally, the second helical section comprises a tapering portion.

Optionally, the first anchor comprises a second helical section, and wherein a cross-sectional dimension of the second helical section is equal to or larger than a cross-sectional dimension of the first portion of the three-dimensional structure.

Optionally, the elongated member is a primary coil, wherein a segment of the primary coil forming first anchor comprises: a stretch-resistance member located within a lumen of the primary coil; or a stiffening coil located within the lumen of the primary coil.

Optionally, the elongated member is a primary coil, wherein a first segment of the primary coil forming the first portion has a first coil-wire diameter, wherein a second segment of the primary coil forming the first anchor has a second coil-wire diameter that is larger than the first coil-wire diameter.

Optionally, the first anchor comprises a third portion comprising third loops lying in different respective third planes that are non-parallel and non-perpendicular with respect to each other.

Optionally, the three-dimensional structure comprises a second anchor at a proximal end of the vaso-occlusive device.

Optionally, the first portion has a cross-sectional dimension that is larger than a cross-sectional dimension of the second portion.

Optionally, the elongated member is a primary coil, and wherein a segment of the primary coil forming the first portion comprises: a stretch-resistance member located within a lumen of the primary coil; or a stiffening coil located within the lumen of the primary coil.

Optionally, the elongated member is a primary coil, wherein a first segment of the primary coil forming the first portion has a first coil-wire diameter, and wherein a second segment of the primary coil forming the second portion has a second coil-wire diameter that is less than the first coil-wire diameter.

Optionally, the three-dimensional structure comprises a third portion having third loops, and a second helical section disposed between the second portion and the third portion; and wherein the third loops comprise at least three third loops lying within at least three different respective third planes that are non-parallel and non-perpendicular with respect to each other.

Optionally, the secondary configuration comprises an elongated configuration configured to occlude a blood vessel.

A vaso-occlusive device includes: an elongated member having a primary configuration when constrained inside a delivery catheter; wherein the elongated member is configured to form a three-dimensional structure having a secondary configuration when deployed outside the delivery catheter; wherein the three-dimensional structure comprises a first portion having first loops, and a second portion having second loops; wherein the first loops comprise a first set of at least three first loops lying within at least three different respective first planes that are non-parallel and non-perpendicular with respect to each other; wherein the second loops comprise a second set of at least three first loops lying within at least three different respective second planes that are non-parallel and non-perpendicular with respect to each other; and wherein the secondary configuration comprises an elongated configuration configured to occlude a blood vessel.

Other and further aspects and features of embodiments of the disclosed inventions will become apparent from the ensuing detailed description in view of the accompanying figures.

### BRIEF DESCRIPTION OF DRAWINGS

The drawings illustrate the design and utility of various aspects of the devices and methods disclosed herein, in which similar elements are referred to by common reference numerals. It should be noted that elements of similar structures or functions are represented by like reference numerals throughout the figures. It should also be noted that the figures are only intended to facilitate the description of the various aspects of the disclosed technology. They are not intended as an exhaustive description of the technology or as a limitation on the scope of the technology, which is defined only by the appended claims and their equivalents. In addition, an illustrated example of the disclosed technology need not have all the aspects or advantages shown or described herein. An aspect or an advantage described in conjunction with a particular example of the disclosed technology is not necessarily limited to that example and can be practiced in any other examples even if not so illustrated or explicitly described. In order to better appreciate how the above-recited and other advantages and objects of the present technology are obtained, a more particular description of the present technology briefly described above will be rendered by reference to specific examples thereof, which are illustrated in the accompanying drawings. With the understanding that these drawings and corresponding description depict only illustrative examples of the disclosed technology and are not therefore to be considered limiting of its scope, the technology will be described and explained with additional specificity and detail through the use of the accompanying drawings in which:

FIG. 1 illustrates a vaso-occlusive device configured to occlude a blood vessel.

FIG. 2 illustrates the vaso-occlusive device of FIG. 1, particularly showing the vaso-occlusive device further having a distal anchor.

FIGS. 3A-3D illustrate different techniques for implementing the distal anchor of FIG. 2.

FIG. 4 illustrates the vaso-occlusive device of FIG. 2, particularly showing the vaso-occlusive device further having a proximal helical section.

FIG. 5 illustrates the vaso-occlusive device of FIG. 4, particularly showing the vaso-occlusive device further having additional portion and additional helical section.

FIGS. 6A-6C illustrate variation of the vaso-occlusive device of FIG. 5.

FIG. 7 is a side, perspective view of a part of the vaso-occlusive device of FIG. 1 in a relaxed, deployed configuration.

FIG. 8A is a top perspective view of a simplification of the intersecting planes of the 3 loops forming a pyramidal portion of a vaso-occlusive device in the shape of a triangular pyramid.

FIG. 8B is a top perspective view of a simplification of the intersecting planes of 4 loops forming a pyramidal portion of a vaso-occlusive device in the shape of a square pyramid.

FIG. 9 is a side view of post for forming a loop of a vaso-occlusive device, particularly illustrating the tapering angle for the loop.

FIG. 10 is a side, perspective view of an example of an alternative pyramidal portion of the vaso-occlusive device of FIG. 1 in which the loops have varying diameters and varying numbers of turns.

FIG. 11A is a side, perspective view of an exemplary mandrel for making a part of the vaso-occlusive device of FIG. 1.

FIG. 11B is a side, perspective view of an exemplary mandrel for making a part of a vaso-occlusive device having 4 loops forming the pyramidal portion in the shape of a square pyramid.

FIG. 12A is a top, perspective view of the mandrel of FIG. 11A with a wire wound around the mandrel to make a part of the vaso-occlusive device of FIG. 1.

FIG. 12B is a side, perspective view of the mandrel of FIG. 12A with the wire wound around the mandrel to make the vaso-occlusive device of FIG. 1.

FIG. 13 is a flow chart showing a method of making the vaso-occlusive devices described herein using the mandrels described herein.

FIG. 14 illustrates an example of a mandrel being utilized to make the vaso-occlusive device of FIG. 5.

FIG. 15 illustrates a prototype of the vaso-occlusive device of FIG. 5.

FIG. 16A illustrates a catheter being utilized to deliver a vaso-occlusive device into a model of a blood vessel

FIG. 16B illustrates a vaso-occlusive device delivered out of a catheter into a model of a blood vessel.

FIG. 17A is a cross-sectional side view of a vaso-occlusive system including the vaso-occlusive device of FIG. 1 in its constrained, delivery configuration within a delivery catheter.

FIG. 17B is a cross-sectional, side view of the vaso-occlusive system of FIG. 17A with the vaso-occlusive device deployed out of the delivery catheter and in its expanded, deployed configuration.

FIG. 18 is a flow chart of an exemplary method of using the vaso-occlusive system of FIGS. 17A and 17B to deploy the vaso-occlusive devices disclosed herein into a blood vessel.

FIG. 19 illustrates another vaso-occlusive device configured to occlude a blood vessel.

FIG. 20 illustrates a variation of the vaso-occlusive device of FIG. 19, particularly showing a portion of the vaso-occlusive device being implemented as an anchor.

FIG. 21 illustrates the vaso-occlusive device of FIG. 20, particularly showing the vaso-occlusive device having a proximal helical section.

FIG. 22 illustrates the vaso-occlusive device of FIG. 21, particularly showing the vaso-occlusive device having additional portions.

FIG. 23 illustrates a variation of the vaso-occlusive device of FIG. 22, particularly showing the vaso-occlusive device having no proximal helical section.

FIG. 24 illustrates an example of a mandrel being utilized to make the vaso-occlusive device of FIG. 22.

FIG. 25 illustrates a prototype of the vaso-occlusive device of FIG. 22.

FIG. 26A illustrates a catheter being utilized to deliver a vaso-occlusive device into a model of a blood vessel

FIG. 26B illustrates a vaso-occlusive device delivered out of a catheter into a model of a blood vessel.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

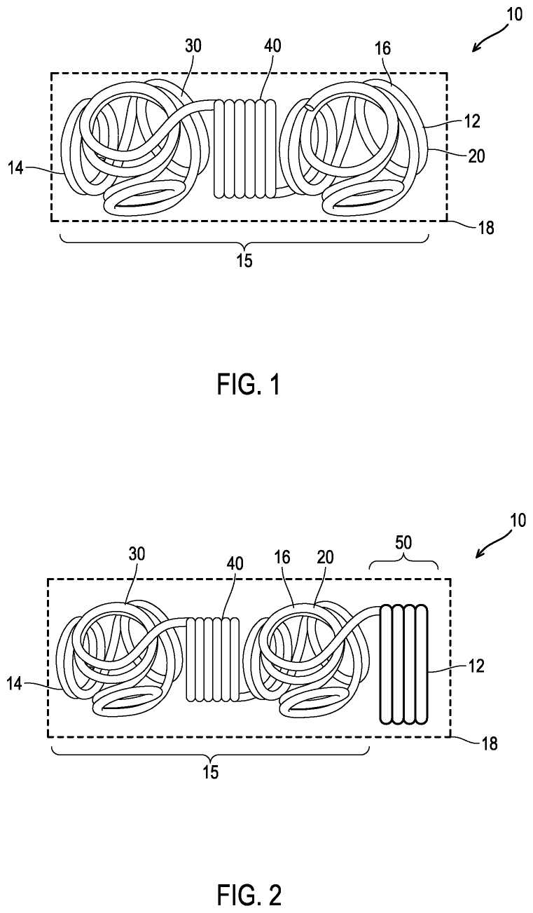

FIG. 1 illustrates a vaso-occlusive device 10 configured to occlude a blood vessel. The vaso-occlusive device 10 is configured for placement in a main lumen of the blood vessel. For example, in some cases, the vaso-occlusive device 10 may be utilized to treat tumor or cancer by blocking blood flow through the blood vessel that supplies blood to the tumor or cancer.

As shown in the figure, the vaso-occlusive device 10 includes an elongated member 16 extending between a distal end 12 and a proximal end 14 of the vaso-occlusive device 10. The terms “distal” and “proximal” as used herein are relative to the vaso-occlusive device 10 as it is intended to be deployed, wherein the term “distal” refers to being situated toward the end of the device 10 which is inserted first, and “proximal” refers to being situated toward the end of the device 10 which is inserted last. The elongated member 16 may be bent or stretched to a primary configuration when constrained inside a delivery catheter. As shown in the figure, the elongated member 16 is configured to form a three-dimensional structure 18 having a secondary configuration when deployed outside the delivery catheter. The three-dimensional structure 18 includes a first portion 20 having first loops, a second portion 30 having second loops, and a first helical section 40 disposed between the first portion 20 and the second portion 30. The first helical section 40 has at least 1.0 turns. In some cases, the first helical section 40 may have a number of turns that is anywhere from 1.0 turns to 20 turns (or more). The number of first loops of the first portion 20 is at least three (four first loops are shown in the example). The at least three first loops lie within at least three different respective first planes that are non-parallel and non-perpendicular with respect to each other. The number of second loops of the second portion 30 is at least three (four second loops are shown in the example). The at least three second loops lie within at least three different respective second planes that are non-parallel and non-perpendicular with respect to each other.

In the illustrated example, the loops of the first portion 20 lie in different respective planes that form a pyramidal shape. The pyramidal shape may be a polyhedral shape formed of a polygonal base having n number of sides and n number of lateral faces connecting to an apex. By means of non-limiting examples, the polyhedral shape may be a tetrahedral shape (triangular base and 3 lateral faces, also referred to as a triangular pyramid), pentahedral shape (quadrilateral base and 4 lateral faces, also referred to as a square or rectangular pyramid), hexahedral shape (pentagonal base and 5 lateral faces, also referred to as a pentagonal pyramid), etc. In the illustrated example, the pyramidal shape formed by the planes associated with the loops of the first portion 20 has a base, and three lateral sides. Alternatively, the pyramidal shape formed by the planes associated with the loops of the first portion may have a base, and four lateral sides. In further cases, the pyramidal shape of the first portion 20 may not include the base. For example, the pyramidal shape of the first portion 20 may include only first loops lying in respective planes that correspond with lateral sides of the pyramidal shape.

Similarly, the loops of the second portion 30 lie in different respective planes that form a pyramidal shape. The pyramidal shape may be a polyhedral shape formed of a polygonal base having n number of sides and n number of lateral faces connecting to an apex. By means of non-limiting examples, the polyhedral shape may be a tetrahedral shape (triangular base and 3 lateral faces, also referred to as a triangular pyramid), pentahedral shape (quadrilateral base and 4 lateral faces, also referred to as a square or rectangular pyramid), hexahedral shape (pentagonal base and 5 lateral faces, also referred to as a pentagonal pyramid), etc. In the illustrated example, the pyramidal shape formed by the planes associated with the loops of the first portion 30 has a base, and three lateral sides. Alternatively, the pyramidal shape formed by the planes associated with the loops of the first portion may have a base, and four lateral sides. In further cases, the pyramidal shape of the second portion 30 may not include the base. For example, the pyramidal shape of the second portion 30 may include only first loops lying in respective planes that correspond with lateral sides of the pyramidal shape.

In the illustrated example, one or each of the loops in the first portion 20 and the second portion 30 is a closed loop. In other cases, one or each of the loops in the first portion 20 and the second portion 30 may be an open loop. In some cases, a loop for the first portion 20 or the second portion 30 may be created by the elongated member 16 forming one or more turns (e.g., 1 turn, 1.5 turn, 2 turns, 3 turns, etc.) for the loop. The loop diameter can be in the range of 0.5 mm to 15 mm, preferably 1 mm to 7 mm. In some cases, the loop diameter may be 90% to 160% of the target vessel diameter.

In some cases, the number of loops of the first portion 20 may be the same as the number of loops of the second portion 30. In other cases, the number of loops of the first portion 20 may be different from the number of loops of the second portion 30. This allows the vaso-occlusive device 10 to provide different filling characteristics (e.g., different packing densities) along a length of the blood vessel.

The first helical section 40 of the vaso-occlusive device 10 is configured to separate the first portion 20 and the second portion 30, and to increase packing efficiency for the vaso-occlusive device 10. In some cases, the first helical section 40 may optionally be configured to set a relative positioning (e.g., orientation) between the first portion 20 and the second portion 30. Also, in some cases, the first helical section may be configured to provide some filing capability for the vaso-occlusive device 10. In the illustrated example, the longitudinal length of the first helical section 40 is shorter than a cross-sectional dimension of the first portion 20 and/or the second portion 30. In other cases, the longitudinal length of the first helical section 40 may be longer than the cross-sectional dimension of the first portion 20 and/or the second portion 30. The first helical section 40 of the vaso-occlusive device 10 may be configured (e.g., by making it longer) to increase the packing density provided by the vaso-occlusive device 10. The first helical section 40 can also prevent tangling between the loops of the first portion 20 with the loops of the second portion 30 when the device is pulled back into a delivery microcatheter.

As shown in the figure, the cross-sectional dimension of the first helical section 40 is less than the cross-sectional dimension of the first portion 20 and the second portion 30. Such configuration is advantageous because it allows the first and second portions 20 and 30 to engage with the walls of the blood vessel at two different longitudinal locations along the blood vessel, while defining a space therebetween to be filled by the first helical section 40. In some cases, if the first helical section 40 is made longer, the first helical section 40 may be folded, bent, twisted, curled up, etc., to occlude at least a part of the lumen of the blood vessel.

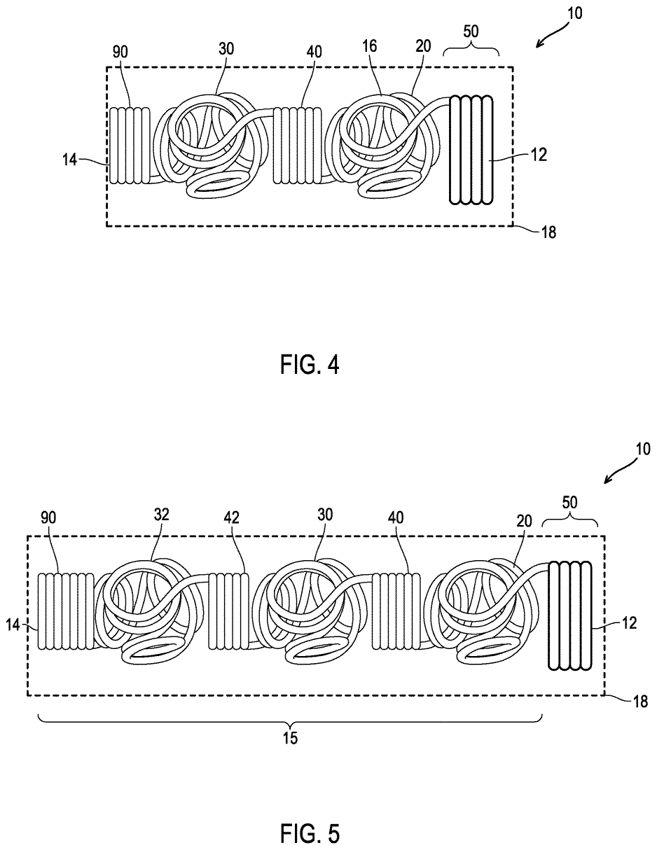

The total length of the entire portion 15 including the first portion 20, the second portion 30, the first helical portion 40, and the proximal helical portion 90 of FIG. 4 is at least 1 cm, preferably at least 5 cm when the device is constrained in a delivery microcatheter.

In the illustrated example. The first portion 20, the second portion 30, and the first helical section 40 form a filling portion 15 for the vaso-occlusive device 10. In some cases, the filling portion 15 has a bending stiffness (EI) that is less than 1.5×10⁻⁶ lb-in², and more preferably less than 1×10⁻⁶ lb-in². This is advantageous because it allows the filling portion 15 to be softer to thereby reduce the risk of rupturing the wall of the blood vessel during or after delivery of the vaso-occlusive device 10. In other cases, the filling portion 15 has a bending stiffness (EI) that is more than 1.5×10⁻⁶ lb-in². Also, in some cases, the filling portion 15 may be soft enough to conform to a shape of the lumen of the blood vessel being occluded, and to form into a dense coil mass. The vaso-occlusive device 10 has an overall elongated configuration, which allows the vaso-occlusive device to more easily occlude a lumen of a blood vessel.

Portions 20 and 30 are configured to provide dense packing and contribute to anchoring of the occlusion plug, while the helical section 40 increases total device length for added occlusion plug density with minimal contribution to delivery friction. The helical section 40 also prevents tangling of the loops between portion 20 and portion 30.

It should be noted that the first portion 20 is not limited to having the configurations described, and that the first portion 20 may have other configurations in other cases. For example, in other cases, the first portion 20 may have loops that form non-pyramidal shapes, such as a square shape, a random shape, etc. Similarly, the second portion 30 is not limited to having the configurations described, and that the second portion 30 may have other configurations in other cases. For example, in other cases, the second portion 30 may have loops that form non-pyramidal shapes, such as a square shape, a random shape, etc.

In the illustrated example, the elongated member 16 is a coil (e.g., a primary coil) formed by a coil wire. The primary coil can have an outside diameter from 0.005″ to 0.030″, preferably from 0.008″ to 0.020″ (e.g., 0.010″, 0.014″, etc.). In some cases, the primary coil may be formed by winding a coil wire with a diameter from 0.0005″ to 0.005″ over a mandrel with a diameter from 0.005″ to 0.025″. For example, the elongated member can be a coil formed by winding a wire diameter of 0.00175″ over a mandrel with a diameter of 0.0102″. The coil wire may have a circular cross-section, or a non-circular cross-section, such as an elliptical section, a polygonal section, etc.

The first portion 20 is formed by the primary coil bending into a number of loops. Similarly, the second portion 30 is formed by the primary coil bending into a number of loops. The first helical section 40 is also formed by the primary coil bending into a number of helical loops. Thus, the first portion 20, the second portion 30, and the first helical section 40 are formed by different respective segments of the elongated member 16. Although not explicitly shown in the figure, it should be understood that there is a transition segment (a part of the elongated member 16) extending or transitioning between first portion 20 and the first helical section 40, and another transition segment (a part of the elongated member 16) extending or transitioning between the first helical section 40 and the second portion 30 of the vaso-occlusive device 10. In other cases, the elongated member 16 may be a wire, a mesh, a braid, etc.

FIG. 2 illustrates the vaso-occlusive device 10 of FIG. 1, particularly showing the vaso-occlusive device 10 further having an anchor 50. The anchor 50 is at the distal end 12 of the vaso-occlusive device 10 and is configured to anchor against an interior wall of a blood vessel to be occluded by the vaso-occlusive device 10. In the illustrated example, the anchor 50 is in a form of a helical section and may be formed by a segment of the elongated member 16. In other cases, the anchor 50 may have other shapes, and/or may be separately connected (e.g., via adhesive, weld, solder, mechanical connector, etc.) to the first portion 20 of the vaso-occlusive device. The anchor 50 has a cross-sectional dimension that is equal to or larger than a maximum cross-sectional dimension of the filling portion 15 of the vaso-occlusive device. Alternatively, or additionally, the anchor 50 may have a compression stiffness (e.g., compression in a direction that is perpendicular to a longitudinal axis of the vaso-occlusive device 10) that is higher than a compression stiffness of the filling portion 15 of the vaso-occlusive device 10. In some cases, the anchor 50 may have a bending stiffness (EI) that is larger than 1.5×10⁻⁶ lb.in², and more preferably larger than 2×10⁻⁶ lb.in², and even more preferably larger than 2.5×10⁻⁶ lb.in². This is advantageous because it allows the anchor 50 to better anchor against the wall of the blood vessel. In other cases, the anchor may have a bending stiffness that is less than 1.5×10⁻⁶ lb.in².

The anchor 50 may be made of a biocompatible metallic material with recoverable strain>0.1%, and preferably >0.5%. The anchor 50 may be made from the same or different material of the portions 20, 30 and the helical section 40 in the filling portion 15. In some cases, the cross-sectional dimension (e.g., diameter) of the anchor 50 may be larger than a cross-sectional dimension of the target vessel to be occluded. For example, the anchor 50 may be 5% larger, or preferably 10% larger, or more preferably 10% to 60% larger than the cross-sectional dimension of the blood vessel to be occluded. This is to ensure that the anchor 50 will sufficiently anchor against the wall of the blood vessel.

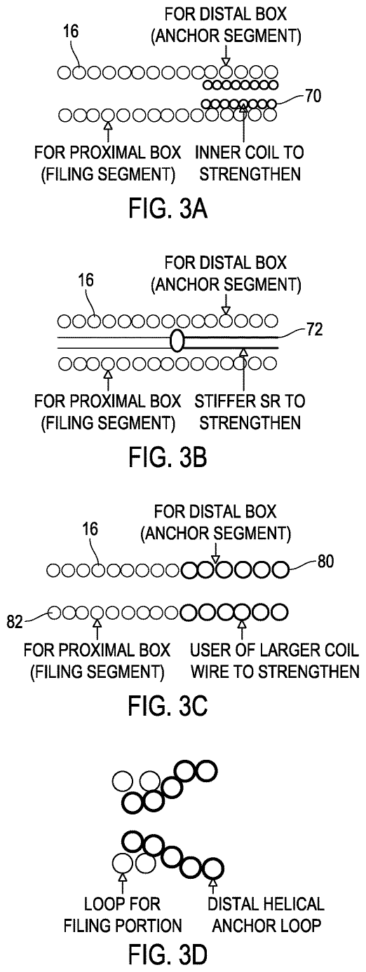

FIGS. 3A-3D illustrate different techniques for implementing the anchor 50 of FIG. 2. As shown in FIG. 3A, the elongated member 16 is a primary coil. In such cases, a segment of the primary coil 16 forming the anchor 50 has a stiffening coil 70 located within the lumen of the primary coil (the elongated member 16). Alternatively, as shown in FIG. 3B, a stretch-resistance member 72 may be provided within a lumen of the primary coil (the elongated member 16) to stiffen the part of the primary coil forming the anchor 50. As shown in FIG. 3C, a first segment 82 of the primary coil forming the first portion 20 may have a first coil-wire diameter, and a second segment 80 of the primary coil forming the first anchor 50 may have a second coil-wire diameter that is larger than the first coil-wire diameter, to thereby provide a stiffer second segment 80 of the primary coil forming the anchor 50. The segments 80, 82 may be parts of the same primary coil. Alternatively, as shown in FIG. 3D, the segments 80, 82 may be different respective coils. In such cases, the segment 80 implementing the anchor 50 may be inserted into the lumen of the coil segment 82 forming the first portion 20. The segments 80, 82 may be welded together or secured to each other using other mechanisms, such as adhesive, or mechanical coupler. In further cases, a segment of the primary coil 16 forming the anchor 50 may be coated by a stiffening material to increase the stiffness of the segment. It should be noted that two or more of the above techniques may be employed together to implement the anchor 50.

FIG. 4 illustrates the vaso-occlusive device 10 of FIG. 2, particularly showing the vaso-occlusive device further having a proximal helical section 90. The proximal helical section 90 is configured to enhance the occlusion capability of the vaso-occlusive device 10. For example, the proximal helical section 90 may be configured to occlude additional part of the lumen of the blood vessel, and/or to increase the packing density for the vaso-occlusive device 10. The proximal helical section 90 is illustrated as being shorter in the longitudinal direction than a cross-sectional dimension of the second portion 30. In other cases, the proximal helical section 90 may be longer than the cross-sectional dimension of the second portion 30. As shown in the figure, the cross-sectional dimension of the proximal helical section 90 is shorter than the cross-sectional dimension of the second portion 30. This configuration allows the proximal helical section 90 to bent, fold, curl up, twist, etc. within a lumen of the blood vessel to occlude the blood vessel.

FIG. 5 illustrates the vaso-occlusive device 10 of FIG. 4, particularly showing the vaso-occlusive device further having an additional portion 32 and an additional helical section 42. In other cases, the vaso-occlusive device 10 may include more than one additional portion 32 (e.g., multiple additional portions 32) and/or more than one additional helical section 42 (e.g., multiple additional helical sections 42). Also, in other cases, the vaso-occlusive device 10 may not include the anchor 50 and/or the proximal helical section 90.

In some cases, the vaso-occlusive device 10 may include a number of pyramidal portions (e.g., portions 20, 30, 32) and a number of helical sections (e.g., helical sections 40, 42), wherein each helical section is disposed between two adjacent pyramidal portions. The number of pyramidal portions may be more than 2, 3, 4, 5, 6, 7, 8, 9, 10, 11, 12, 13, 14, 15, 16, 17, 18, 19, 20, 21, 22, 23, 24, 25, 26, 27, 28, 29, or 30. The number of portions may be selected to fit a particular application. For example, higher number of portions may be selected to fill a larger blood vessel, to achieve a higher occlusion, and/or to achieve a longer occluded segment along the blood vessel.

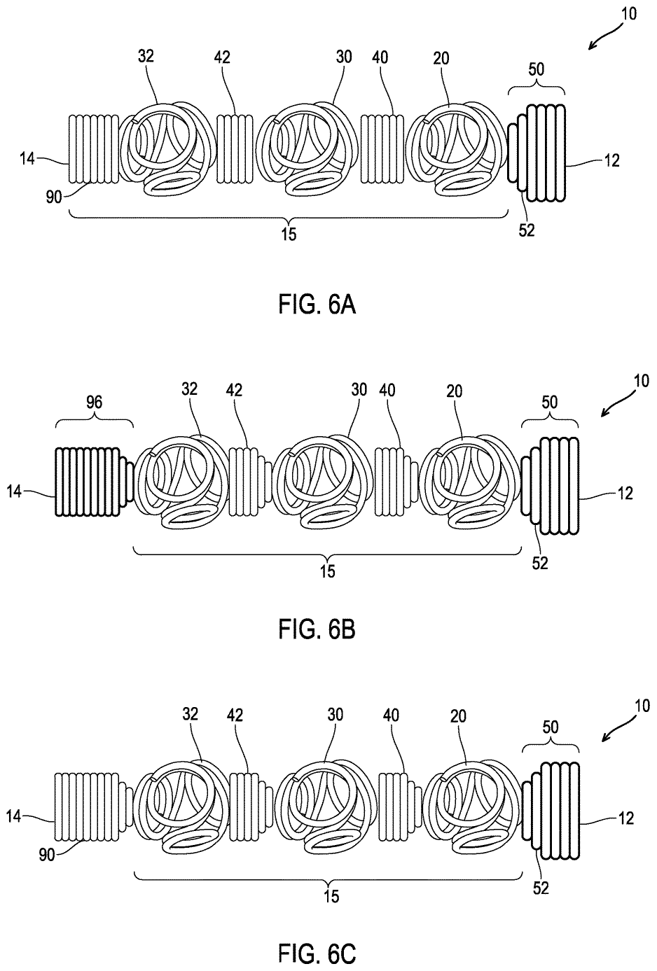

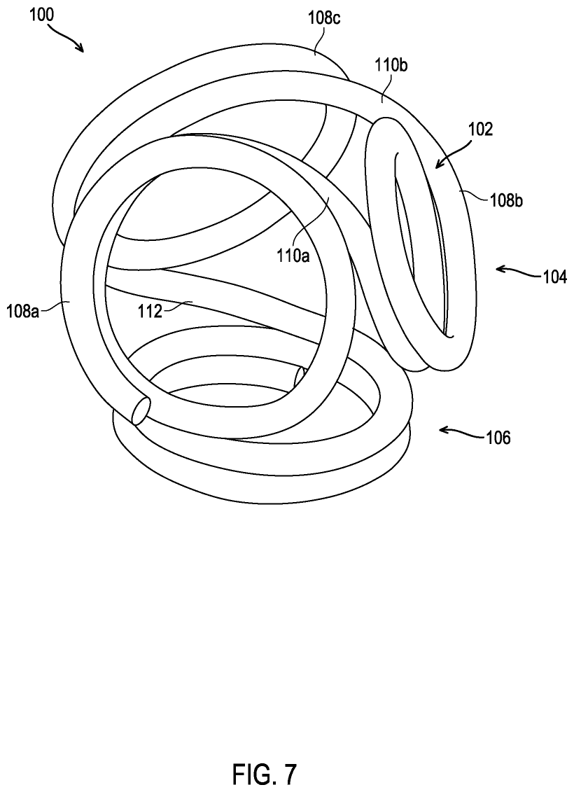

FIGS. 6A-6C illustrate variations of the vaso-occlusive device 10 of FIG. 5. As shown in FIG. 6A, the anchor 50 of the vaso-occlusive device 10 may be implemented as a helical section having a tapering portion 52. The tapering portion 52 is advantageous because it may prevent a part of the first portion 20 from entering into a lumen of the helical section forming the anchor 50. In some cases, as shown in FIG. 3D, the tapering section 52 of the anchor portion 50 may enter the lumen of the loops forming the first portion 20. When this happens, it creates an interlock between the helical anchor segment 50 and the first portion 20 to enhance anchorability.

As shown in FIG. 6B, instead of having the proximal helical section 90 that is configured to fill a lumen of a blood vessel, the vaso-occlusive device 10 may include an anchor 96 at a proximal end 14 of the vaso-occlusive device 10. The anchor 96 may be implemented using any of the techniques of FIGS. 3A-3C. The anchor 96 is configured to improve an anchoring capability of the vaso-occlusive device 10. Providing two anchors 50, 96 at opposite ends of the vaso-occlusive device 10 is advantageous because they reduce the risk of the vaso-occlusive device 10 migrating along the blood vessel and provide two fixed anchoring positions that define a space to be occluded by the filling portion 15 of the vaso-occlusive device. As shown in FIG. 6B, each of the anchors 50, 96 has a tapering portion. The tapering portion is advantageous because it may prevent a part of the portion 20/32 from entering into a lumen of the helical section forming the anchor 50/96. In some cases, as shown in FIG. 3D, the tapering section 52 of the distal anchor portion 50 may enter the lumen of the loops forming the first portion 20. Similarly, the tapering section of the proximal anchor portion 96 may enter the lumen of the loops forming the portion 32. When this happens, it creates a distal interlock between the distal anchor portion 50 and the first portion 20 and a proximal interlock between the portion 32 and the proximal anchor portion 96. Both distal and proximal interlocks enhance the anchorability of the occlusion device 10 within the vessel. Although the helical section 40/42 is illustrated as having a tapering portion at one end, in other cases, the helical section 40/42 may have tapering portions at opposite ends. In other cases, one or both of the anchors 50, 96 may not have any tapering portion. In such cases, the anchor 50/96 may have a cross-sectional dimension that is uniform along a majority or an entirety of a length of the anchor. Also, as shown in FIG. 6B, the helical sections 40, 42 in the filler portion 15 have respective tapering portions. In other cases, each of the helical sections 40, 42 in the filler portion 15 may have a cross-sectional dimension that is uniform along a majority or an entirety of a length of the helical section.

As shown in FIG. 6C, in some cases, the vaso-occlusive device 10 may include a proximal helical section 90 (instead of the anchor 90) at the proximal end 14 of the vaso-occlusive device 10. The helical section 90 may include a tapering portion like that shown in the figure. The tapering portion may prevent a part of the portion 32 from entering into the lumen of the proximal helical section 90. In other cases, the helical section 90 may have a cross-sectional dimension that is uniform along a majority or an entirety of a length of the helical section. The helical section 90 may have a length that is equal to, shorter or longer than, a cross-sectional dimension of the portion 20/30/32. For example, in some cases, the helical section 90 may have a length that is 0.5, 1, 1.5, 2, 3, 4, 5, 6, 7, 8, 9, 10, etc. times the cross-sectional dimension of the portion 20/30/32.

It should be noted that in the examples of FIGS. 2 and 4-6C, the anchor 50 has a cross-sectional dimension (i.e. outside diameter) that is larger than that of the filling portion 15 (e.g., larger than the portion 20/30/32). In some cases, the cross-sectional dimension of the anchor 50 may be 5% larger, or 10% larger, or 20% larger than the cross-sectional dimension of the portion 20/30/32. Also, the portions 20, 30, 32 in the filling portion 15 may have the same size, or may have different respective sizes. In some cases, the size of the portions 20, 30, 32 in the vaso-occlusive device 10 of FIG. 2 and 4-6C may decrease from the distal end towards the proximal end. Thus, the cross-sectional dimension of the portion 20 may be larger than the cross-sectional dimension of the portion 30, and the cross-sectional dimension of the portion 30 may be larger than the cross-sectional dimension of the portion 32.

As described, the portion 20/30 of the vaso-occlusive device 10 may be a pyramidal portion. FIG. 7 illustrates an example of a pyramidal portion 104 of a vaso-occlusive device 100. The vaso-occlusive device 100 may be any of the vaso-occlusive devices 10 described herein, and the pyramidal portion 104 may be an example of the portion 20, 30, 32, etc. In particular, FIG. 7 depicts a portion of the vaso-occlusive device 100 in its secondary configuration in a relaxed, unconstrained condition. In other words, FIG. 7 shows the portion of the vaso-occlusive device 100 when there are no external forces exerting on the portion of the vaso-occlusive device 100. The secondary configuration of the portion of the vaso-occlusive device 100 has a pyramidal portion 104 and a helical section 106. The helical section 106 may be an example of the first helical section 40 described herein. In other cases, the vaso-occlusive device 100 may not include the helical section 106.

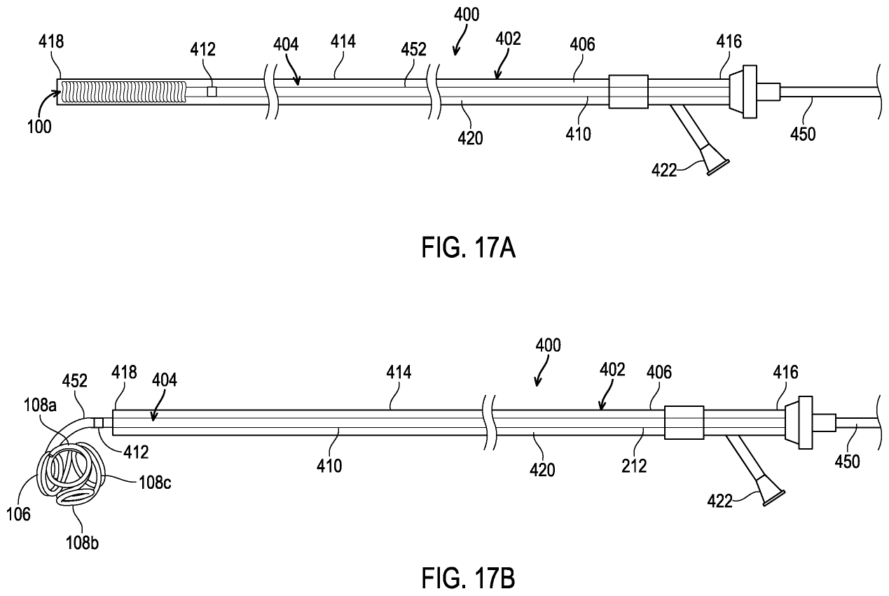

The vaso-occlusive 100 comprises a wire 102 having a primary configuration in a constrained configuration. The wire 102 may be the elongated member 16 described herein. For instance, the constrained configuration may be an elongate, helical coil when the wire 102 is constrained within a delivery catheter (see FIG. 17A). The primary shape may have a longitudinal length anywhere between 1 cm to 100 cm. The wire 102 is made from any suitable material, such as a radiopaque, shape memory material. By means of non-limiting examples, the material of the wire 102 may be platinum group metals, including platinum, rhodium, palladium, and rhenium, as well as tungsten, gold, silver, tantalum, and alloys of these metals. In one specific example, the wire 102 may be made from Pt-8% W. In some cases, the material of the wire 102 may have a yield strain>0.1%, and preferably >0.5%. Also, the material of the wire 102 may have a tensile strength that is larger than 150 Ksi, with elongation larger than 1% to ensure manufacturability. In other cases, the material of the wire 102 may have other mechanical properties that are different from the above examples.

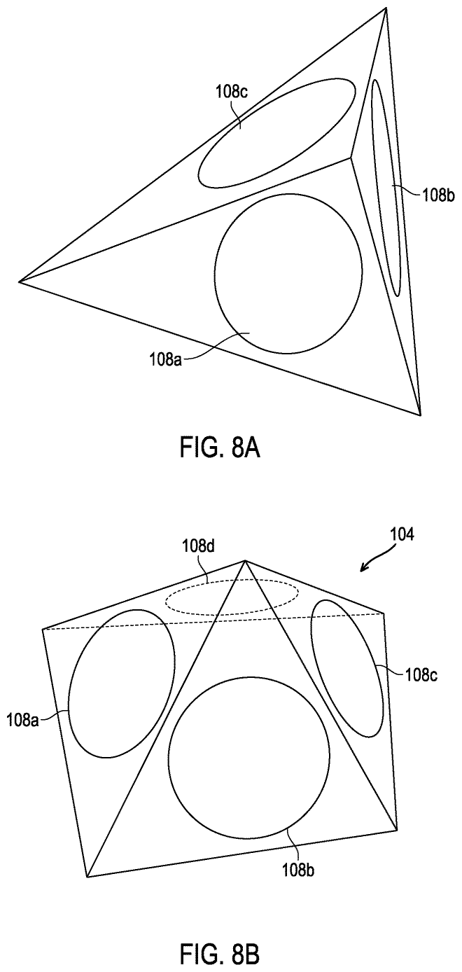

The pyramidal portion 104 of the vaso-occlusive device 100 includes a plurality of loops 108 wound from the wire 102. In the illustrated example of FIG. 7, the pyramidal portion 104 includes three loops 108a, 108b, and 108c lying in respective planes corresponding to three lateral sides of a pyramidal shape. The pyramidal portion 104 does not include any loop lying in a plane corresponding with the base of the pyramidal shape. However, in other cases, the pyramidal portion 104 may include a fourth loop lying in a plane corresponding with the base of the pyramidal shape. The respective planes in which the loops 108 lie are non-parallel, non-perpendicular, and intersect with each other. As depicted in FIG. 8A, a simplification of the intersecting planes of the loops 108 forms a tetrahedral, more specifically, a triangular pyramid. The vaso-occlusive device 100 of FIG. 7 is an example and is not limited to loops 108 forming a triangular pyramid shape, but may be other suitable pyramidal shapes, such as n number of loops 108 forming a pyramid having a polygonal base having n number of sides and n number of lateral faces connecting to an apex. For instance, the vaso-occlusive device 100 may have loops 108 in each lateral face of a pyramidal shape which may be a square pyramid having a square base and 4 lateral faces, a quadrilateral pyramid having a quadrilateral base and 4 lateral faces, a pentagonal pyramid having a pentagonal base and 5 lateral faces, etc. As depicted in FIG. 8B, the pyramidal portion 104 may have 4 loops 108 in each lateral face arranged to form a square pyramid having a square base and 4 lateral faces.

Turning back to FIG. 7, each loop 108 includes a winding forming a closed loop of greater than 360°, i.e., at least a complete, closed loop. In the illustrated vaso-occlusive device 100, each loop 108 includes a winding of 1⅔ loops (also referred to as “turns”), which is equivalent to a winding of 600°. Alternatively, each loop 108 may have at least 1⅔ turns, or between 1⅔ turns (600°) and 2 turns (720°).

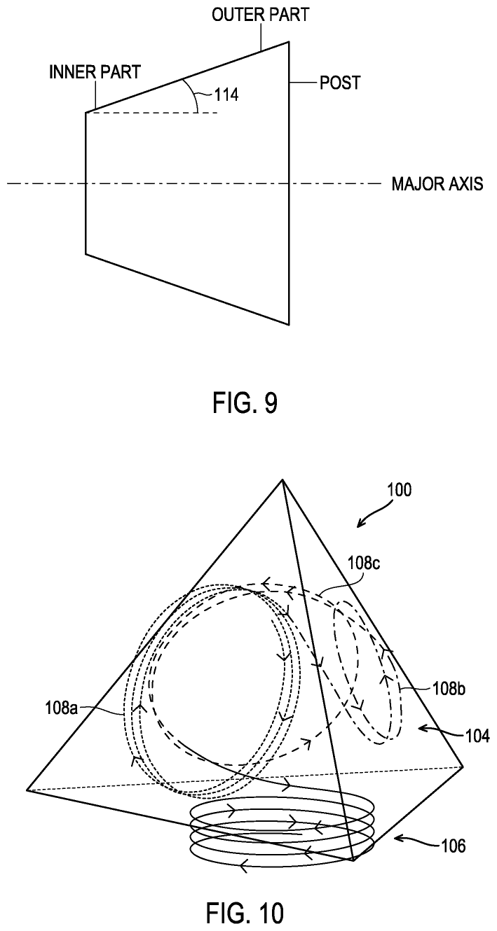

The winding of each of the loops 108 also has a width which tapers outwardly from a reference point of the interior of the pyramidal portion. In the case of a helical coil as depicted in the example of FIG. 7, the width of each loop 108 is the diameter of the turn. If the loops 108 are wound in a different shape, such as a square, pentagon, or other polygonal or curved shape, the width may be an average width, such an average of the maximum and minimum width drawn through the geometric center of the shape. Turning the example of FIG. 7, the diameter of each loop 108 tapers such that the diameter of the outer part of the loop 108 is larger than the diameter of the inner part of the loop 108. This may be better explained with reference to a cross-section of a cylindrical post of a mandrel upon which the wire 102 is wound to form a loop 108, as shown in FIG. 9. The post has a diameter which tapers outwardly from the inner part of the post to the outer part of the post, as depicted in FIG. 9. The angle 114 of the post taper defines the angle of the taper of the loop 108 in the secondary configuration of the vaso-occlusive device 100. Due to the outwardly tapering diameter, the turns of each of the loops 108 is inward facing. In other words, the surface of the loops faces inward toward the geometric interior of the pyramidal shape of the pyramidal portion 104. Thus, in some embodiments, each loop has more than one turn that increases in size (e.g., hoop diameter) such that the hoop diameter of a turn closer to a periphery of the pyramidal portion is larger than the hoop diameter of a turn further away from the periphery of the pyramidal portion. In other cases, each loop may have more than one turn having a same size (e.g., hoop diameter).

Each of the loops 108 has a diameter (defined as the diameter of the inner most turn of each loop 108) between 90 to 160 percent of a diameter of a blood vessel the vaso-occlusive device 100 is designed to occlude. Preferably, the diameter of each loop 108 may be between 100 to 130 percent of a diameter of a blood vessel the vaso-occlusive device 100 is designed to occlude.

In an alternative design of the vaso-occlusive device 100, the loops 108 may have differing diameters and/or differing numbers of turns of the wire (e.g., the wire 102 of FIG. 7), as illustrated in FIG. 10. As shown in FIG. 10, the first loop 108a has a larger diameter and more turns of the wire 102 (3 turns, i.e., 1080°) than the second loop 108 b which has 1⅔ turns (600°). The third loop 108c also has a larger diameter than the second loop 108b (same diameter as the first loop 108a and 1⅔ turns (600°).

The pyramidal portion 104 also includes a respective transition segment 110 of the wire 102 which is between and connecting each adjacent loop 108 to each other. Each transition segment 110 extends from the ending of one loop 108 to the beginning of the subsequent loop 108. Hence, the transition segment 110a extends from the ending of the first loop 108a to the beginning of the second loop 108b, and the transition segment 110b extends from the ending of the second loop 108s to the beginning of the third loop 108c.

There is also a transition segment 112 from the ending of the third loop 108c winding to the beginning of the helical section 106, which may be an example of the first helical section 40. The helical section 106 comprises a winding of the wire 102 which extends proximally from the pyramidal shape 104. The helical section 106 may extend from the imaginary base of the pyramidal shape of the pyramidal portion 104. In the illustrated example of FIG. 7, the helical section 106 has a constant diameter. Alternatively, the helical section 106 may have a diameter which tapers outwardly or inwardly as it extends from the proximally from the pyramidal portion 104. The helical section 106 comprises at least one full loop of the wire 102. The helical section 106 does not have a maximum degree of winding and may include any suitable number of turns, such as between 1 and 200 turns. The helical section 106 depicted in FIG. 7 has about 2 full turns of the wire 102. The helical section 106 depicted in FIG. 10 has about 4 full turns of the wire 102.

In other cases, the vaso-occlusive device 100 may not include the helical section 106. Also, in further cases, the vaso-occlusive device 100 may include an additional pyramidal portion 100. In such cases, the transition segment 112 may extend from the third loop 108c (or a last loop of the first pyramidal portion 100) to a first loop of the additional pyramidal portion 100. Furthermore, in other cases, the pyramidal portion 100 may include a loop forming the base of the pyramidal portion 100. In such cases, the transition segment 112 transitions to the loop forming the base of the pyramidal portion 100.

The pyramidal shape formed by the loops 108 of the vaso-occlusive device 100 has an inherent ability to effectively dissipate forces applied to the vaso-occlusive device 100, thereby reducing the risk of imparting excessive forces on the walls of the blood vessel which could rupture the blood vessel. Furthermore, the loops 108 are tapered to be inwardly facing and comprise full, closed loops and crossing, overlapping segments which fold more tightly than open loops when deployed in a blood vessel, thereby reducing the risk of herniation during placement. At the same time, shape stability is not compromised because of the inherent ability of the pyramidal shape to dissipate forces applied at the narrow end of the pyramidal shape (e.g., corresponding with the apex of the pyramidal shape). Furthermore, as illustrated in FIG. 7, the pyramidal shaped vaso-occlusive device 100 is more compact than a cube shaped vaso-occlusive device having 8 coils forming the 8 sides of a cube. In other words, the volume of the pyramidal shaped vaso-occlusive device 100 has a smaller volume than a cube.

FIG. 8A is a top perspective view of a simplification of the intersecting planes of the 3 loops of FIG. 7 forming the pyramidal portion of the vaso-occlusive device 10 in the shape of a triangular pyramid. FIG. 8B is a top perspective view of a simplification of the intersecting planes of 4 loops forming the pyramidal portion of a vaso-occlusive device 10 in the shape of a square pyramid.

It should be noted that the loops of a portion (e.g., portion 20, 30, 32, etc.) of the vaso-occlusive device 10 may have the same size and same number of turns. In other cases, two or more of the loops of a portion (e.g., portion 20, 30, 32, etc.) of the vaso-occlusive device 10 may have different respective sizes and/or different respective numbers of turns. FIG. 10 is a side, perspective view of an example of an alternative pyramidal portion of the vaso-occlusive device 10 of FIG. 1 in which the loops have varying diameters and varying numbers of turns.

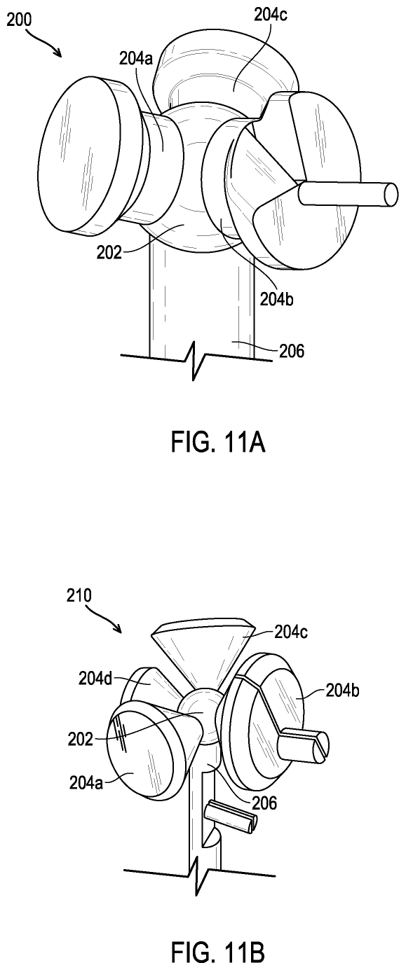

Turning to FIG. 11A, an exemplary mandrel 200 for making a portion (e.g., portion 20, 30, 32, etc.) of the vaso-occlusive device 10 using a method 300 (described below) is illustrated. The mandrel 200 is used to wind the wire 102 (which may be an example of the elongated member 16) around to form the secondary configuration of the vaso-occlusive device 10. The mandrel 200 is configured to form a vaso-occlusive device 10 comprising a pyramidal portion having a triangular pyramid shape.

The mandrel 200 has a central, spherical element 202. A plurality of posts 204 extend outwardly from the central spherical element 202. The posts 204 are configured to form the loops of a portion (e.g., portion 20, 30, 32, etc.) of the vaso-occlusive device 10. The posts 204 are spaced angularly around the spherical element 202. In the depicted mandrel 200, the posts 204 are evenly spaced such that a respective longitudinal axis of each loop is evenly, angularly spaced around the spherical element 202. Accordingly, in the case of three posts 204, the posts 204 are spaced apart by 120°.

Each post 204 has a cross-sectional diameter which tapers outwardly as the post 204 extends away from the spherical element 202, as shown in FIG. 9 which shows a side view of one of the posts 204. The longitudinal axis of each post 204 is also canted distally such that respective planes perpendicular to each respective longitudinal axis are non-parallel and non-perpendicular and the planes have an intersection which forms a pyramidal shape having an apex distal to the posts 204. In other cases, each post 204 may not have a taper configuration, and may instead have a constant cross-sectional dimension.

The mandrel 200 also has a body post 206 extending from the spherical element 202. The body post 206 is configured to form a helical section (e.g., helical section 40) of the vaso-occlusive device 100. In the illustrated example of FIG. 11A, the body post 206 extends proximally from the spherical element 202. The body post 206 may be a cylinder having a constant diameter, or alternatively, the body post 206 may taper outwardly or inwardly as it extends from the spherical element 202.

The mandrel 200 includes additional set(s) of posts 204 (not shown) for forming an additional pyramidal portion of the vaso-occlusive device 100. In particular, the mandrel 200 may include a first set of posts 204 (like that shown in FIG. 11A), and a second set of posts 204 that are separated from the first set of posts 204 by the body post 206.

With the assistance of the present description, one of ordinary skill in the art would appreciate how to modify the mandrel 200 to be configured to make a vaso-occlusive device 10 comprising a pyramidal portion having the other pyramid shapes disclosed herein. For instance, FIG. 11B shows a mandrel 210 which is configured to make a vaso-occlusive device 10 comprising a pyramidal portion having a square pyramid shape. The mandrel 210 is same or substantially similar to the mandrel 200, except that the mandrel 210 has four posts 204 evenly, angularly spaced around the central, spherical member 202.

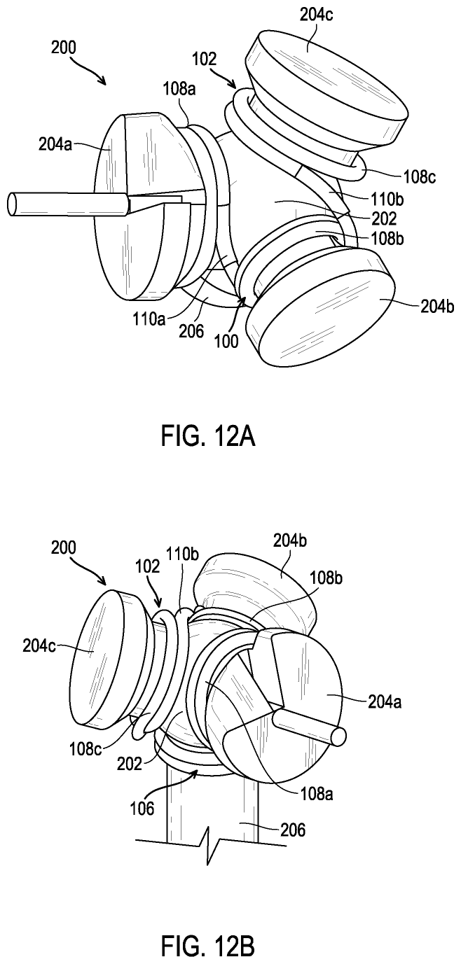

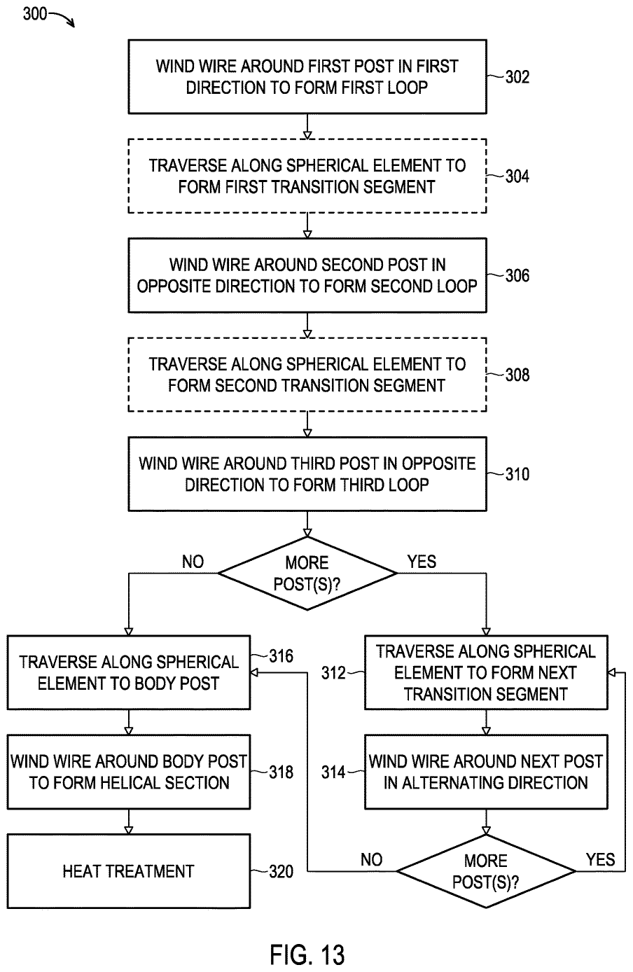

Turning to the illustrations in FIGS. 12A-12B and the flow chart of FIG. 13, an exemplary method 300 of making a part (e.g., portion 20, 30, 32, etc.) of a vaso-occlusive device 10 using the mandrel 200 will now be described. A wire 102 (which may be an example of the elongated member 16) having a primary configuration is wound around the mandrel 200. As described herein, the wire 102 may be formed of a shape memory material, and the primary configuration may be a configuration of the wire 102 in a constrained condition, such as within a sheath or delivery catheter. The primary configuration of the wire 102 may have any suitable shape, such as a helical coil—in which case, the wire 102 may be a primary coil.

At step 302, the wire 102 is first wound around the first post 108a in a first direction starting on the first post 108a and towards the intersection of the first post 204a, and the spherical element 202. Subsequent windings start at the intersection of the post 204 and spherical element 202 and wind away from the spherical element 202. As used herein, a winding “direction” is relative to viewing inwardly along the winding axis toward the position of the attachment of a winding post to the central, spherical element or other central structure. Thus, the “first direction” as shown in the example of FIGS. 12A and 12B is a clockwise direction. The wire 102 is wound around the first post 204a to form the desired degree of winding for the first loop 108a, as described herein.

After winding the wire 102 around the first post 204a, at step 304, the wire 102 is traversed along the spherical element 202 forming the first transition segment 110a to the second post 108b immediately adjacent to the first post 108a. At step 306, the wire 102 is wound around the second post 204b in a second direction opposite to the first direction (counterclockwise as shown in the example of FIGS. 12A-12B) starting at the intersection of the second post 204b and the spherical element 202. The wire 102 is wound around the second post 204b to form the desired degree of winding for the second loop 108b, as described herein.

After winding the wire 102 around the second post 204b, at step 308, the wire 102 is traversed along the spherical element 202 forming the second transition segment 110b to the third post 204c immediately adjacent to the second post 204b. At step 310, the wire 102 is wound around the third post 204c in a third direction opposite to the second direction (clockwise as shown in the example of FIGS. 12A-12B) starting at the intersection of the third post 204c and the spherical element 202. The wire 102 is wound around the third post 204c to form the desired degree of winding for the third loop 108c, as described herein.

Looking at a post 204 that is being wound axially along the axis of rotation, each of the three other posts 204 can be described at ⅓ rotations as the wire 102 is being wound. Accordingly, a winding of 1⅔ loops or turns (i.e., 600°) means that as the wire 102 is wound around the post 204, each of the other posts 204, 206 that are passed represents a winding of ⅓ of a loop or turn. Hence, a winding of 1⅔ loops passes the three other posts 204, 206 once, and then re-passes 2 of the 3 other posts 204, 206 for a total of 1⅔ loops. A winding of about 2⅔ (960°) passes each of the other posts 204, 206 twice and then passes the next 2 posts a third time. The winding of a full loop (360°), or multiple full loops (360°×a whole number) plus another ⅔ of a loop, positions the end of a loop 108 to wind in the opposite direction on the next adjacent post 204.

In the case of a mandrel 200 having more than 3 posts 204, at step 312, the wire is 102 traversed along the spherical element 202 in a next transition segment 110 to a next post 204 immediately adjacent to the preceding post 204. At step 314, the wire is wound around the next post 204 to form the next loop 108, and steps 312-314 are repeated for each additional post 204. After winding the wire around the last of the posts 204, at step 316, the wire 102 is transitioned along the spherical element 202 to the body post 206.

At step 318, the wire 102 is wound around the body post 206 to form the desired degree of winding for the helical section (e.g., helical section 40), as described herein. In some cases, if the vaso-occlusive device 10 does not include the helical section 40, then the mandrel 200 may not include the body post 206, and the method 300 may not include step 318.

In some cases, if the vaso-occlusive device 10 has another pyramidal portion, after step 314, steps 302-314 may be repeated using an additional set of posts 204 to form the other pyramidal portion.

At step 320, the wire 102 is heat treated to set the secondary configuration of the vaso-occlusive device 10 as wound on the mandrel 200. The wire 102 may then be removed from the mandrel 200 after heat treatment, and the wire 102 will take on the secondary configuration having the secondary shape as wound on the mandrel in its relaxed, unconstrained condition.

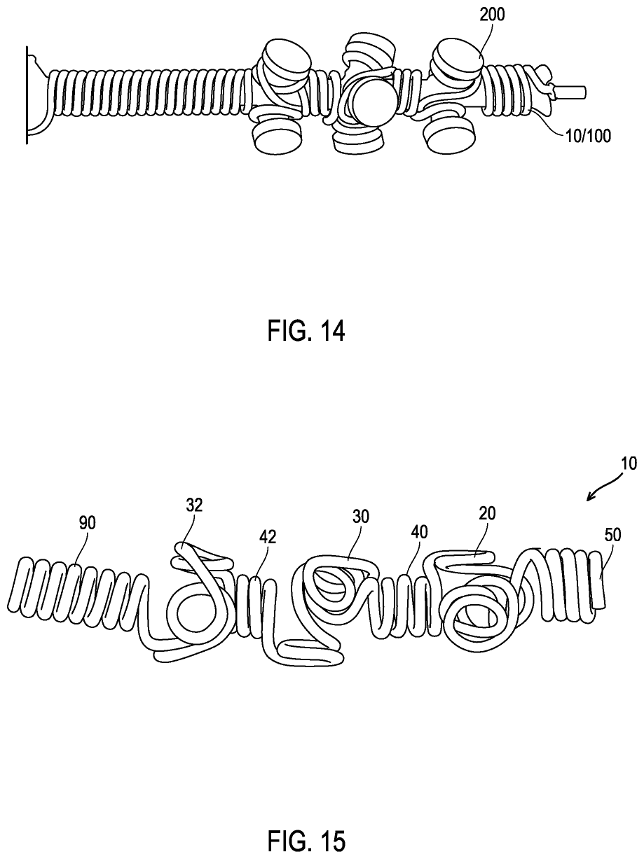

FIG. 14 illustrates an example of a mandrel 200 being utilized to make the vaso-occlusive device 10 of FIG. 5. FIG. 15 illustrates a prototype of the vaso-occlusive device 10 of FIG. 5. In the prototype, the anchor 50 is formed from a first segment of a primary coil made from Pt-8W. The primary coil's dimensions are 0.00175″×0.0102″ (i.e., the coil is formed by winding a 0.00175″ wire over a 0.0102″ mandrel). The anchor 50 also includes an inner coil (which is an example of the inner coil 70 described with reference to FIG. 3A) with the dimensions 0.00225″×0.005″ (i.e., the coil is formed by winding a 0.00225″ wire over a 0.005″ mandrel). The anchor 50 has a helical shape, with a cross-sectional dimension of 2.25 mm, and a longitudinal length of 4 cm. The first portion 20, the second portion 30, and the third portion 32 of the prototype are formed from a second segment, a third segment, and a fourth segment, respectively, of the primary coil. Each of the first portion 20, the second portion 30, and the third portion 32 has a cross-sectional dimension of 2 mm. In the illustrated example, the total longitudinal length of all portions including the first portion 20, the second portion 30, and the third portion 32 is 10 cm. The helical sections 40, 42 are formed from a fifth segment and a sixth segment, respectively, of the primary coil. Each of the helical sections 40, 42 has a cross-sectional dimension of 2 mm and a longitudinal length of 3 cm. The proximal helical section 90 is formed from a seventh segment of the primary coil. The proximal helical section 90 has a cross-sectional dimension of 2 mm, and a longitudinal length of 8 cm.

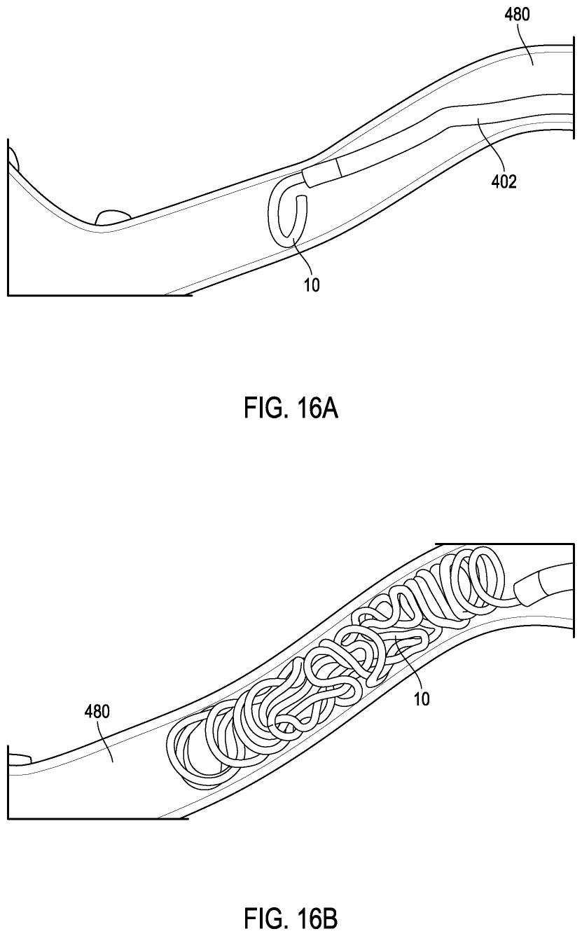

FIG. 16A illustrates a catheter 402 being utilized to deliver the prototype of the vaso-occlusive device 10 of FIG. 15 into a model of a blood vessel 480. FIG. 16B illustrates the vaso-occlusive device 10 delivered out of the catheter 402 into a model of a blood vessel 480. As shown in the figure, the vaso-occlusive device 10 forms an elongated packed structure that at least partly occludes the blood vessel. The vessel shown in the FIG. 16 has an internal diameter (ID) of 2 mm and the delivered compact coil mass (i.e. dense coil mass) is expected to be around 12 mm. It should be pointed out that the vaso-occlusive device 10 can be used to occlude the vessel with ID ranging from 1.5 mm and 2.5 mm and the resulting dense coil mass is expected to be between 5 mm and 20 mm.

FIG. 17A is a cross-sectional side view of a vaso-occlusive system including a vaso-occlusive device 100 (which may be any of the vaso-occlusive devices 10 described herein) in its constrained, delivery configuration within a delivery catheter. FIG. 17B is a cross-sectional, side view of the vaso-occlusive system of FIG. 17A with the vaso-occlusive device 100 deployed out of the delivery catheter and in its expanded, deployed configuration. As shown in FIGS. 17A-17B, the vaso-occlusive device 100 may be a component of a vaso-occlusive system 400 which can be used to deploy the vaso-occlusive device 100 into a body cavity, such as a main lumen of a blood vessel. The vaso-occlusive system 400 comprises a delivery assembly 402 and a vaso-occlusive assembly 404. As shown in FIGS. 17A and 17B, the delivery assembly 402 may include a delivery catheter 406 and an optional guidewire (not shown). The vaso-occlusive assembly 404 comprises a vaso-occlusive device 100 and a pusher member 410 detachably coupled to the vaso-occlusive device 100 via a detachment device or junction 412. FIG. 17A shows the vaso-occlusive assembly 404 after it has been slidably disposed within the delivery catheter 406 such that the vaso-occlusive device 100 is in its compact, delivery configuration.

The delivery catheter 406 is an elongated, flexible tube, and can be, for example, a microcatheter or the like. The delivery catheter 406 comprises an elongate sheath body 414 having a proximal portion 416, a distal portion 418 and a lumen 420 extending from the proximal portion 416 to the distal portion 418. The proximal portion 416 of the delivery catheter 406 typically remains outside of the patient and accessible to the clinician when the vaso-occlusive system 400 is used, while the distal portion 418 is sized and dimensioned to reach remote locations of a patient's vasculature and is configured to deliver the vaso-occlusive device 100 to a body cavity such as a main lumen of a blood vessel. In some cases, the delivery catheter 406 may be a microcatheter having a lumen with a cross-sectional dimension that is less 0.05 inch (e.g., 0.02 inch, 0.017 inch, etc.). The delivery catheter 406 may also have one or more ports 422 in fluid communication with the lumen 420 for introducing into, or removing fluids from, the sheath body 414. The sheath body 414 may be composed of suitable polymeric materials, metals and/or alloys, such as polyethylene, stainless steel or other suitable biocompatible materials or combinations thereof. In some instances, the proximal portion 416 may include a reinforcement layer, such as a braided layer or coiled layer to enhance the pushability of the sheath body 414. The sheath body 414 may include a transition region between the proximal portion 416 and the distal portion 418.

The vaso-occlusive assembly 404 also comprises a pusher member 410. The pusher member 410 is configured to be slidably received within the lumen 420 of the delivery catheter 406. The pusher member 410 has a proximal portion 450, which extends proximal of the proximal portion 416 of the delivery catheter 406, and a distal portion 452 which is detachably coupled to the proximal end of the vaso-occlusive device 100 via the detachment device 412. The pusher member 410 may be a coil, wire, tendon, guidewire, torquable cable tube, hypotube, or the like, having a sufficient columnar strength to permit pushing of the vaso-occlusive device 100 out through distal end 418 of the delivery catheter 406 and into a main lumen of a blood vessel. In some cases, the vaso-occlusive device 100 may be smoothly delivered through the catheter 406 via a pushing force that is <=0.1 lb, and preferably <=0.06 lb. Accordingly, the total frictional force between the vaso-occlusive device 100 and the inner wall of the catheter 406 is less than 0.1 lb, and preferably less than 0.06 lb.

The detachment device 412 provides a detachable connection between the pusher member 410 and the vaso-occlusive device 100. The detachment device 412 may comprise an electrolytically detachment, mechanical connector, heat activated detachment, dissolving detachment, or other mechanical, thermal and hydraulic mechanism. For instance, the detachment device 412 may be an electrolytically degradable segment for electrolytically decoupling the vaso-occlusive device 100 from the pusher member 410.

In some cases, if the system includes the optional guidewire, the optional guidewire may be positioned within the patient's vasculature with the distal end of the guidewire located at the target insertion site (e.g., a main lumen of a blood vessel), the delivery catheter 406 is advanced over the guidewire with the guidewire disposed within the lumen 420 of the delivery catheter 406. In a “rapid-exchange” configuration of the delivery catheter 406 and guidewire, the guidewire extends through only a distal portion of the delivery catheter 406, such as a rapid-exchange lumen. The guidewire may be used by first advancing the guidewire through the patient's vasculature to the target insertion site and then advancing the delivery catheter 406 over the guidewire to the target insertion site.

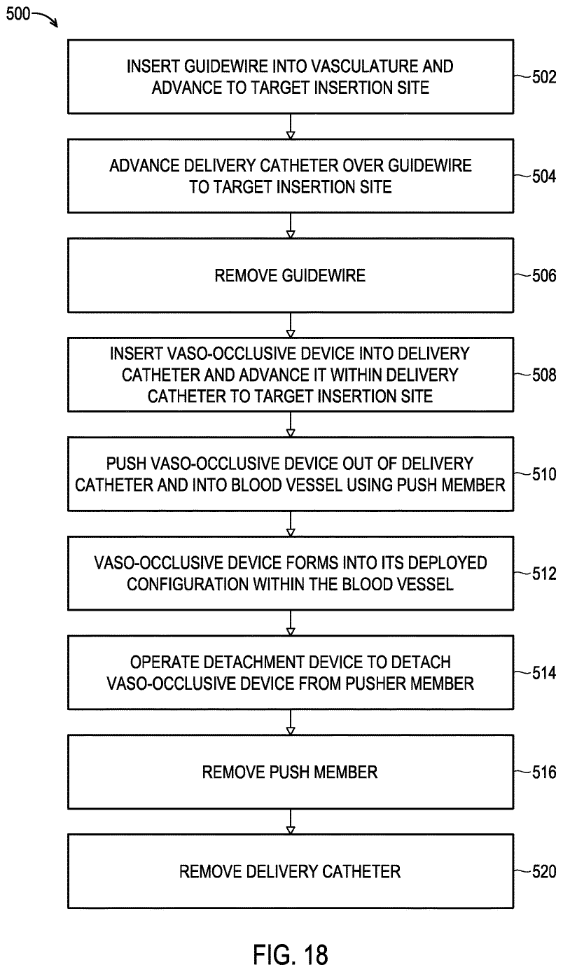

FIG. 18 is a flow chart of an exemplary method 500 of using the vaso-occlusive system of FIGS. 17A and 17B to deploy the vaso-occlusive device 10 disclosed herein into a blood vessel. The method 500 will be described with respect to deploying the vaso-occlusive device 10 into a blood vessel, as an example. However, the method 500 is not limited to deploying the vaso-occlusive device 10 into a blood vessel but may be used to deploy the vaso-occlusive device 10, or other medical device as disclosed herein, into any suitable anatomical cavity which is accessible via a patient's vasculature. Referring to the flow chart of FIG. 18, at step 502, a guidewire is inserted into the patient's vasculature and is advanced to the target insertion site, namely a blood vessel. As described herein, the use of the guidewire is optional, and is not required in the method 500 of using the vaso-occlusive system 400 to deploy the vaso-occlusive device 10.

At step 504, the delivery catheter 406 of the delivery assembly 402 is advanced over the guidewire until it is positioned with the open distal end 418 proximal a target location inside a lumen of a blood vessel. At step 506, the guidewire is pulled out of the delivery catheter 406 leaving the delivery catheter 406 in position. At step 508, the vaso-occlusive assembly 404 is inserted into the delivery catheter 406 of the delivery assembly 402 and advanced within the delivery catheter 406 to position the distal end of the vaso-occlusive device 10 adjacent the distal portion 418 of the delivery catheter 406. At this position, the proximal portion 450 of the pusher member 410 remains proximal and outside of the proximal portion 416 of the delivery catheter 406. Prior to inserting the vaso-occlusive device 10 into the delivery catheter 406, the vaso-occlusive device 10 may be pre-installed in a sheath such that the vaso-occlusive device 10 is in its constrained, delivery configuration (primary configuration). The vaso-occlusive device 10 is then inserted into the delivery catheter 406 by placing a distal end of the sheath in abutment with the proximal end 416 of the delivery catheter 406 and extruding the vaso-occlusive device 10 from the sheath into the delivery catheter 406 such that the vaso-occlusive device 10 remains in its delivery configuration within the delivery catheter 406.

At step 510, the vaso-occlusive device 10 is pushed through the lumen 420 of the delivery catheter 406 and distally out of the delivery catheter 406 into a blood vessel by pushing on the proximal portion 450 of the pusher member 410. At step 512, as the vaso-occlusive device 10 is pushed out of the open distal end 418 of the delivery catheter 406, various parts (e.g., portions 20, 30, 32, and helical sections 40, 42, 90) of the vaso-occlusive device 10 are advanced into the blood vessel and formed in the blood vessel. As a result, the vaso-occlusive device 100 in its delivery configuration (primary configuration) expands into the secondary configuration (deployed configuration). In some cases, if the vaso-occlusive device 10 includes the anchor 50 at the distal end 12, the anchor 50 is deployed first out of the delivery catheter 406, and anchors against a wall of the blood vessel.

Once the entire vaso-occlusive device 10 is inserted into the main lumen of the blood vessel, at step 514, the detachment device 412 is actuated, activated or otherwise operated to detach the vaso-occlusive device 10 from the pusher-member 410. At step 516, the pusher member 410 is removed from the patient's vasculature by withdrawing it out through the delivery catheter 406. In some cases, a single vaso-occlusive device 10 may be sufficient to occlude the blood vessel, and the method 500 then proceeds to step 520 in which the delivery catheter 406 is removed from the patient's vasculature. In other cases, if the single vaso-occlusive device 10 is not sufficient to occlude the blood vessel, then additional vaso-occlusive device(s) 10 may be deployed. If multiple vaso-occlusive devices 10 are being implanted, then the process of steps 508-516 are repeated to deliver a sufficient number of vaso-occlusive devices 10 to occlude the blood vessel. After the sufficient number of vaso-occlusive devices 10 are implanted in the blood vessel, the delivery catheter 406 is removed at step 520.

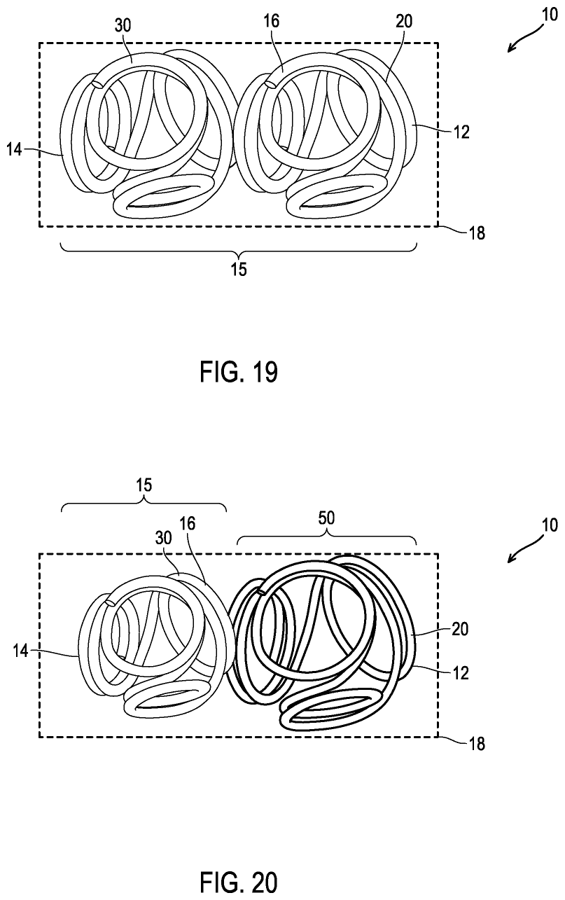

FIG. 19 illustrates another vaso-occlusive device 10 configured to occlude a blood vessel. The vaso-occlusive device 10 is the same as that described with reference to FIG. 1, except that the vaso-occlusive device 10 does not include the first helical section 40 between the first portion 20 and the second portion 30. In particular, as shown in the figure, the vaso-occlusive device 10 includes an elongated member 16 extending between a distal end 12 and a proximal end 14 of the vaso-occlusive device 10. The elongated member 16 may be bent or stretched to a primary configuration when constrained inside a delivery catheter. As shown in the figure, the elongated member 16 is configured to form a three-dimensional structure 18 having a secondary configuration when deployed outside the delivery catheter. The three-dimensional structure 18 includes a first portion 20 having first loops, and a second portion 30 having second loops. The number of first loops of the first portion 20 is at least three (four first loops are shown in the example). The at least three first loops lie within at least three different respective first planes that are non-parallel and non-perpendicular with respect to each other. The number of second loops of the second portion 30 is at least three (four second loops are shown in the example). The at least three second loops lie within at least three different respective second planes that are non-parallel and non-perpendicular with respect to each other.

In the illustrated example, the loops of the first portion 20 lie in different respective planes that form a pyramidal shape. The pyramidal shape may be a polyhedral shape formed of a polygonal base having n number of sides and n number of lateral faces connecting to an apex. By means of non-limiting examples, the polyhedral shape may be a tetrahedral shape (triangular base and 3 lateral faces, also referred to as a triangular pyramid), pentahedral shape (quadrilateral base and 4 lateral faces, also referred to as a square or rectangular pyramid), hexahedral shape (pentagonal base and 5 lateral faces, also referred to as a pentagonal pyramid), etc. In the illustrated example, the pyramidal shape formed by the planes associated with the loops of the first portion 20 has a base, and three lateral sides. Alternatively, the pyramidal shape formed by the planes associated with the loops of the first portion may have a base, and four lateral sides. In further cases, the pyramidal shape of the first portion 20 may not include the base. For example, the pyramidal shape of the first portion 20 may include only first loops lying in respective planes that correspond with lateral sides of the pyramidal shape.

Similarly, the loops of the second portion 30 lie in different respective planes that form a pyramidal shape. The pyramidal shape may be a polyhedral shape formed of a polygonal base having n number of sides and n number of lateral faces connecting to an apex. By means of non-limiting examples, the polyhedral shape may be a tetrahedral shape (triangular base and 3 lateral faces, also referred to as a triangular pyramid), pentahedral shape (quadrilateral base and 4 lateral faces, also referred to as a square or rectangular pyramid), hexahedral shape (pentagonal base and 5 lateral faces, also referred to as a pentagonal pyramid), etc. In the illustrated example, the pyramidal shape formed by the planes associated with the loops of the first portion 30 has a base, and three lateral sides. Alternatively, the pyramidal shape formed by the planes associated with the loops of the first portion may have a base, and four lateral sides. In further cases, the pyramidal shape of the second portion 30 may not include the base. For example, the pyramidal shape of the second portion 30 may include only first loops lying in respective planes that correspond with lateral sides of the pyramidal shape.

In the illustrated example, the elongated member 16 is a coil (e.g., a primary coil) formed by a coil wire. The coil wire may have a circular cross-section, or a non-circular cross-section. The first portion 20 is formed by the primary coil bending into a number of loops. Similarly, the second portion 30 is formed by the primary coil bending into a number of loops. Thus, the first portion 20 and the second portion 30 are formed by different respective segments of the elongated member 16. Although not explicitly shown in the figure, it should be understood that there is a transition segment (a part of the elongated member 16) extending or transitioning between first portion 20 and the second portion 30 of the vaso-occlusive device 10. In other cases, the elongated member 16 may be a wire, a mesh, a braid, etc.

It should be noted that the first portion 20 is not limited to having the configurations described, and that the first portion 20 may have other configurations in other cases. For example, in other cases, the first portion 20 may have loops that form non-pyramidal shapes, such as a square shape, a random shape, etc. Similarly, the second portion 30 is not limited to having the configurations described, and that the second portion 30 may have other configurations in other cases. For example, in other cases, the second portion 30 may have loops that form non-pyramidal shapes, such as a square shape, a random shape, etc.

FIG. 20 illustrates a variation of the vaso-occlusive device of FIG. 19, particularly showing the portion 20 of the vaso-occlusive device 10 being implemented as an anchor 50. As shown in the figure, the anchor 50 is at the distal end 12 of the vaso-occlusive device 10 and is configured to anchor against an interior wall of a blood vessel to be occluded by the vaso-occlusive device 10. In the illustrated example, the anchor 50 has a pyramidal shape, and is formed by a segment of the elongated member 16. In other cases, the anchor 50 may have other shapes, and/or may be separately connected (e.g., via adhesive, weld, solder, mechanical connector, etc.) to the first portion 20 of the vaso-occlusive device. The anchor 50 has a cross-sectional dimension that is equal to or larger than a maximum cross-sectional dimension of the filling portion 15 of the vaso-occlusive device. Alternatively, or additionally, the anchor 50 may have a compression stiffness (e.g., compression in a direction that is perpendicular to a longitudinal axis of the vaso-occlusive device 10) that is higher than a compression stiffness of the filling portion 15 of the vaso-occlusive device 10. The anchor 50 may be implemented using any of the techniques, or any combination of the techniques, described with reference to FIGS. 3A-3C.

The anchor 50 may be made of a biocompatible metallic material with recoverable strain>0.1%, and preferably >0.5%. The anchor 50 may be made from the same or different material for the portion 30 in the filling portion 15. In some cases, the cross-sectional dimension (e.g., diameter) of the anchor 50 may be larger than a cross-sectional dimension of the target vessel to be occluded. For example, the anchor 50 may be 5% larger, or preferably 10% larger, or more preferably 10% to 60% larger than the cross-sectional dimension of the blood vessel to be occluded. This ensure that the anchor 50 will sufficiently anchor against the wall of the blood vessel.

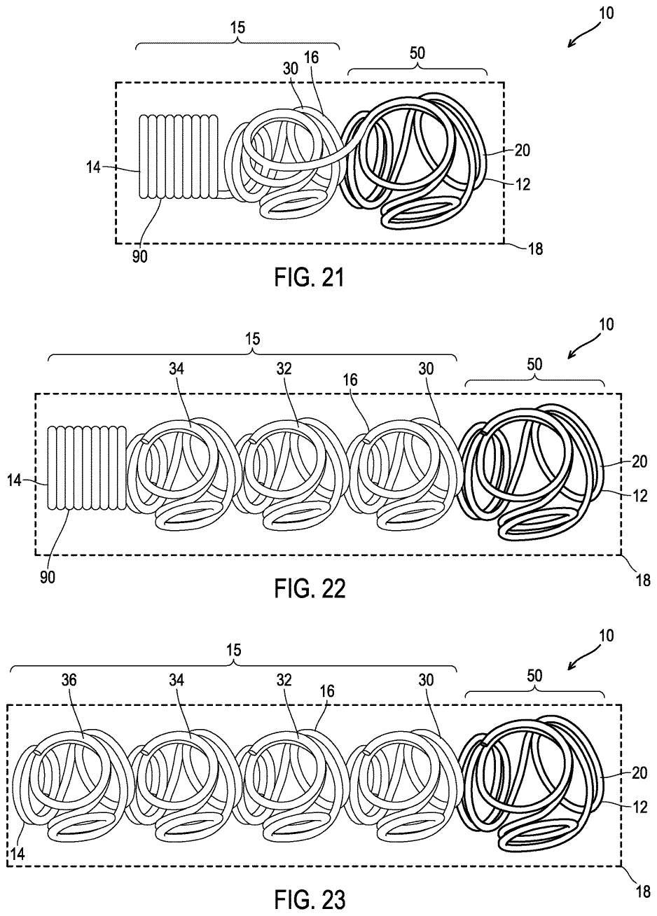

FIG. 21 illustrates the vaso-occlusive device of FIG. 20, particularly showing the vaso-occlusive device having a proximal helical section 90. The proximal helical section 90 is configured to enhance the occlusion capability of the vaso-occlusive device 10. For example, the proximal helical section 90 may be configured to occlude additional part of the lumen of the blood vessel, and/or to increase a packing density for the vaso-occlusive device 10. The proximal helical section 90 is illustrated as being shorter in the longitudinal direction than a cross-sectional dimension of the second portion 30. In other cases, the proximal helical section 90 may be longer than the cross-sectional dimension of the second portion 30. As shown in the figure, the cross-sectional dimension of the proximal helical section 90 is shorter than the cross-sectional dimension of the second portion 30. This configuration allows the proximal helical section 90 to bent, fold, curl up, twist, etc. within a lumen of the blood vessel to occlude the blood vessel.

FIG. 22 illustrates the vaso-occlusive device of FIG. 21, particularly showing the vaso-occlusive device having additional portions 32, 34. In other cases, the vaso-occlusive device 10 may include only one additional portion or more than two additional portions. In some cases, the vaso-occlusive device 10 may include a number of portions (e.g., portions 20, 30, 32). The number of portions may be more than 2, 3, 4, 5, 6, 7, 8, 9, 10, 11, 12, 13, 14, 15, 16, 17, 18, 19, 20, 21, 22, 23, 24, 25, 26, 27, 28, 29, or 30. The number of portions may be selected to fit a particular application. For example, higher number of portions may be selected to fill a larger blood vessel, to achieve a higher occlusion, and/or to achieve a longer occluded segment along the blood vessel. Also, in some cases, the vaso-occlusive device 10 may not include the anchor 50 and/or the proximal helical section 90.

In any of the exemplary vaso-occlusive devices 10 described herein, the proximal helical section 90 is optional, and the vaso-occlusive device 10 may not include the proximal helical section 90. FIG. 23 illustrates a variation of the vaso-occlusive device of FIG. 22, particularly showing the vaso-occlusive device having no proximal helical section. In such cases, the filling portion 15 of the vaso-occlusive device 10 includes the portions 30, 32, 34, 36, which are configured to occlude a main lumen of a blood vessel.

It should be noted that in the examples of FIGS. 20-23, the anchor 50 has a cross-sectional dimension that is larger than that of the filling portion 15 (e.g., larger than the portion 30/32/34). In some cases, the cross-sectional dimension of the anchor 50 may be 5% larger, or 10% larger, or 20% larger than the cross-sectional dimension of the portion 30/32/34. Also, the portions 30, 32, 34, 36 in the filling portion 15 may have the same size, or may have different respective sizes. In some cases, the size of the portions 30, 32, 34, 36 in the vaso-occlusive device 10 of FIG. 23 may decrease from the distal end towards the proximal end. Thus, the cross-sectional dimension of the portion 30 may be larger than the cross-sectional dimension of the portion 32, and the cross-sectional dimension of the portion 32 may be larger than the cross-sectional dimension of the portion 34, and the cross-sectional dimension of the portion 34 may be larger than the cross-sectional dimension of the portion 36.

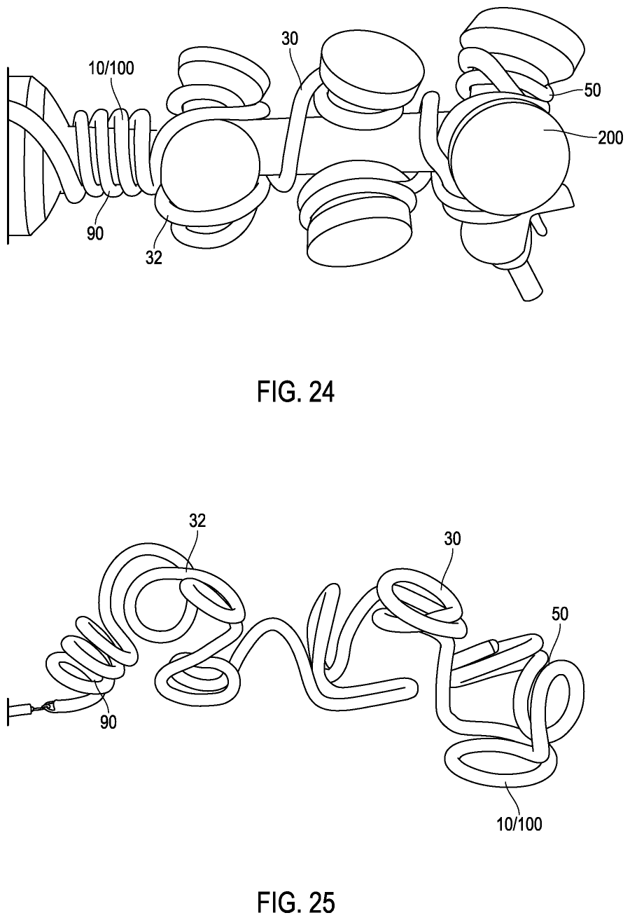

FIG. 24 illustrates an example of a mandrel 200 being utilized to make a vaso-occlusive device 10 that is similar to that shown in FIG. 22. In particular, the vaso-occlusive device 10 being made using the mandrel 200 has the same configuration as that of FIG. 22, except that it does not include the portion 34. FIG. 25 illustrates a prototype of the vaso-occlusive device 10 formed from the mandrel 200 of FIG. 24. In the prototype, the anchor 50 implemented by the first portion 20 is formed from a first segment of a primary coil made from Pt-8W. The primary coil's dimensions are 0.00175″×0.0102″ (i.e., the coil is formed by winding a 0.00175″ wire over a 0.0102″ mandrel). The anchor 50 also includes an inner coil (which is an example of the inner coil 70 described with reference to FIG. 3A) with the dimensions 0.00225″×0.005″ (i.e., the coil is formed by winding a 0.00225″ wire over a 0.005″ mandrel). The anchor 50 has a cross-sectional dimension (i.e., outside diameter) of 2.5 mm, and a longitudinal length (i.e., coil length) of 4 cm. The second portion 30 and the third portion 32 of the prototype are formed from a second segment and a third segment, respectively, of the primary coil. The second portion 30 has a cross-sectional dimension of 2.25 mm, and the third portion 32 has a cross-sectional dimension of 2 mm. The total length of the second portion 30 and the third portion 32 is 6.5 cm. The proximal helical section 90 is formed by a fourth segment of the primary coil, and has a cross-sectional dimension of 2 mm, and a longitudinal length of 4 cm.

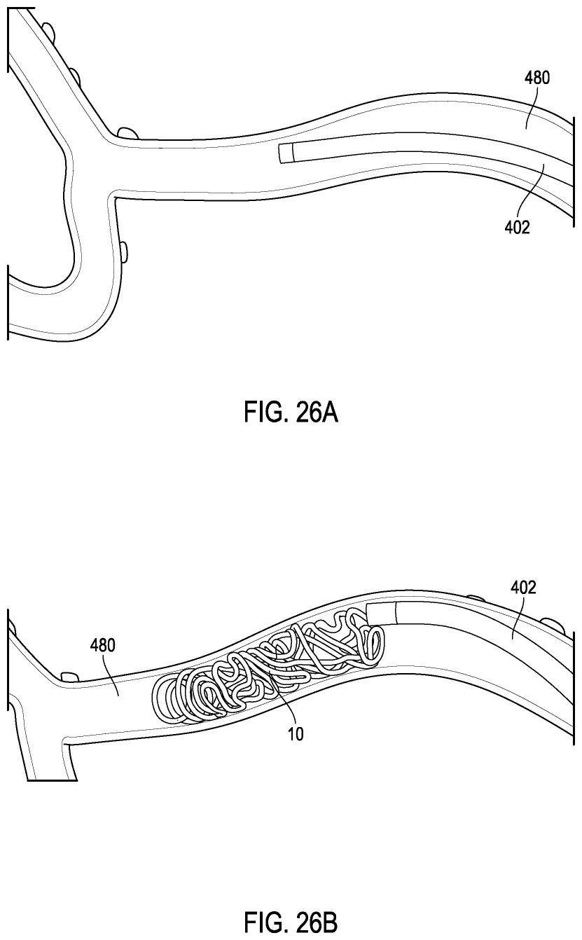

FIG. 26A illustrates a catheter 402 being utilized to deliver the prototype of the vaso-occlusive device 10 of FIG. 25 into a model of a blood vessel 480. FIG. 26B illustrates the vaso-occlusive device 10 delivered out of the catheter 402 into a model of a blood vessel 480. As shown in the figure, the vaso-occlusive device 10 forms an elongated packed structure that efficiently occludes the blood vessel. The vessel shown in the FIG. 26 has an internal diameter (ID) of 2 mm and the delivered compact coil mass (i.e. dense coil mass) is expected to be around 10 mm. It should be pointed out that the vaso-occlusive device 10 can be used to occlude the vessel with ID ranging from 1.5 mm and 2.5 mm and the resulting dense coil mass is expected to be between 5 mm and 15 mm.

The vaso-occlusive devices 10 described herein are advantageous because they are specifically designed to occlude blood vessels. In some cases, the vaso-occlusive device 10 may be utilized to occlude a blood vessel in the neural vasculature and/or in the peripheral vasculature. The vaso-occlusive device 10 may also be utilized for venous vessel occlusion. In some cases, the vaso-occlusive device 10 includes one or more anchors (e.g., anchor 50 and/or anchor 96) designed specifically to engage with the tubular wall of the blood vessel, thereby preventing the vaso-occlusive device 10 from migrating along the lumen of the blood vessel. In addition, the vaso-occlusive device 10 may have a length that is longer than those designed for aneurysm treatment. For example, in some cases, the vaso-occlusive device 10 may have a delivery length (when confined inside a catheter) that is 2 times, 3 times, 4 times, 5 times, or more, compared to that of the delivery length of a vaso-occlusive device designed for aneurysm treatment. This is advantageous because a single vaso-occlusive device 10 may establish a complete or desirable vessel occlusion. Use of a single vaso-occlusive device 10 reduces treatment time and also reduces the risk that one or more vaso-occlusive devices may migrate away from the occlusion site if multiple vaso-occlusive devices are used.

It should be noted that the vaso-occlusive device 10 described herein should not be limited to having the configurations described in the examples, and that the vaso-occlusive device 10 may have other configurations. For example, in other cases, any of the vaso-occlusive devices 10 described herein may include one or more segments with complex shapes, such as a 3D portion having six or more loops (e.g., eight loops). Such 3D portion, for example, may replace the second portion 30 in the vaso-occlusive device 10 of FIG. 1, or may replace the proximal helical section 90 in the vaso-occlusive device of FIGS. 4, 5, 6A, or 6C, or may replace the second portion 30 in the vaso-occlusive device 10 of FIG. 19 or 20, or may replace the proximal helical section 90 in the vaso-occlusive device 10 of FIG. 21 or 22.

In some cases, any of the vaso-occlusive devices 10 described herein may optionally further include fibers and/or surface features to enhance thrombogenicity. The fibers may be mechanically attached to a surface of the elongated member 16 or may be weaved through the coil windings of the primary coil (elongated member 16).

In further cases, any of the vaso-occlusive devices 10 described herein may optionally further include an accelerating agent coated on a surface of the vaso-occlusive device 10 to accelerate the formation of thrombus.

Although the vaso-occlusive devices 10 disclosed herein have been described as being configured for implantation in a lumen of a blood vessel, the vaso-occlusive devices 10 may alternatively be configured for implantation in other cavities in a patient. Thus, the examples of the vaso-occlusive device 10 described herein are not limited to being configured and used for treating blood vessel, but they are particularly well-suited for such configuration and use.

Although particular embodiments of the disclosed inventions have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments of the disclosed inventions shown and described herein are intended to cover alternatives, modifications, and equivalents of the disclosed inventions, which may be included within the scope of the appended claims.

1. A vaso-occlusive device, comprising:
an elongated member having a primary configuration when constrained inside a delivery catheter;
wherein the elongated member is configured to form a three-dimensional structure having a secondary configuration when deployed outside the delivery catheter;
wherein the three-dimensional structure comprises a first portion having first loops, a second portion having second loops, and a first helical section disposed between the first portion and the second portion, and wherein the first helical section comprises at least 1.0 turns;
wherein the first loops of the first portion comprise a first set of at least three first loops lying within at least three different respective first planes that are non-parallel and non-perpendicular with respect to each other; and
wherein the second loops of the second portion comprise a second set of at least three second loops lying within at least three different respective second planes that are non-parallel and non-perpendicular with respect to each other.

2. The vaso-occlusive device of claim 1, wherein the first planes form a pyramidal shape.

3. The vaso-occlusive device of claim 2, wherein the pyramidal shape comprises a polyhedral shape formed of a polygonal base having n number of sides and n number of lateral faces connecting to an apex.

4. The vaso-occlusive device of claim 1, wherein the three-dimensional structure comprises a second helical section extending from the second portion.

5. The vaso-occlusive device of claim 4, wherein the second helical section has a longitudinal length that is longer than a cross-sectional width of the second portion of the three-dimensional structure.

6. The vaso-occlusive device of claim 1, wherein the elongated member is a primary coil.

7. The vaso-occlusive device of claim 6, wherein the first helical section comprises a first secondary coil formed by a first segment of the primary coil, wherein the first portion is formed by a second segment of the primary coil, and the second portion is formed by a third segment of the primary coil.

8. The vaso-occlusive device of claim 1, wherein the three-dimensional structure comprises a first anchor at a distal end of the vaso-occlusive device.

9. The vaso-occlusive device of claim 8, wherein the first anchor comprises a second helical section.

10. The vaso-occlusive device of claim 1, wherein the first anchor comprises a second helical section, and wherein a cross-sectional dimension of the second helical section is equal to or larger than a cross-sectional dimension of the first portion of the three-dimensional structure.

11. The vaso-occlusive device of claim 8, wherein the elongated member is a primary coil, wherein a segment of the primary coil forming first anchor comprises:
a stretch-resistance member located within a lumen of the primary coil; or
a stiffening coil located within the lumen of the primary coil.

12. The vaso-occlusive device of claim 8, wherein the elongated member is a primary coil, wherein a first segment of the primary coil forming the first portion has a first coil-wire diameter, wherein a second segment of the primary coil forming the first anchor has a second coil-wire diameter that is larger than the first coil-wire diameter.

13. The vaso-occlusive device of claim 8, wherein the first anchor comprises a third portion comprising third loops lying in different respective third planes that are non-parallel and non-perpendicular with respect to each other.

14. The vaso-occlusive device of claim 8, wherein the three-dimensional structure comprises a second anchor at a proximal end of the vaso-occlusive device.

15. The vaso-occlusive device of claim 1, wherein the first portion has a cross-sectional dimension that is larger than a cross-sectional dimension of the second portion.

16. The vaso-occlusive device of claim 15, wherein the elongated member is a primary coil, and wherein a segment of the primary coil forming the first portion comprises:
a stretch-resistance member located within a lumen of the primary coil; or
a stiffening coil located within the lumen of the primary coil.

17. The vaso-occlusive device of claim 15, wherein the elongated member is a primary coil, wherein a first segment of the primary coil forming the first portion has a first coil-wire diameter, and wherein a second segment of the primary coil forming the second portion has a second coil-wire diameter that is less than the first coil-wire diameter.

18. The vaso-occlusive device of claim 1, wherein the three-dimensional structure comprises a third portion having third loops, and a second helical section disposed between the second portion and the third portion; and
wherein the third loops comprise at least three third loops lying within at least three different respective third planes that are non-parallel and non-perpendicular with respect to each other.

19. The vaso-occlusive device of claim 1, wherein the secondary configuration comprises an elongated configuration configured to occlude a blood vessel.

20. A vaso-occlusive device, comprising:
an elongated member having a primary configuration when constrained inside a delivery catheter;
wherein the elongated member is configured to form a three-dimensional structure having a secondary configuration when deployed outside the delivery catheter;
wherein the three-dimensional structure comprises a first portion having first loops, and a second portion having second loops;
wherein the first loops comprise a first set of at least three first loops lying within at least three different respective first planes that are non-parallel and non-perpendicular with respect to each other;
wherein the second loops comprise a second set of at least three first loops lying within at least three different respective second planes that are non-parallel and non-perpendicular with respect to each other; and
wherein the secondary configuration comprises an elongated configuration configured to occlude a blood vessel.
